(19) United States
(12) Patent Application Publication
(10) Pub. No.: US 2001/0034019 A1
(43) Pub. Date: Oct. 25, 2001

(54) Chimeric HCV/GBV-B viruses

(76) Inventors: Zhi Hong, Aliso Viejo, CA (US); Nancy J. Butkiewicz, Plainfield, NJ (US); Weidong Zhong, Laguna Niguel, CA (US); Paul Ingravallo, Staten Island, NY (US); Jacquelyn Wright-Minogue, Kenilworth, NJ (US); Johnson YN Lau, Newport Beach, CA (US); Stanley M. Lemon, Galveston, TX (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/742,659

(22) Filed: Dec. 21, 2000

Related U.S. Application Data
(60) Provisional application No. 60/171,469, filed on Dec. 22, 1999.

(51) Int. Cl. C12Q 1/70; A61K 49/00; C07H 21/04; C12N 5/06; C12N 7/01

(52) U.S. Cl. 435/5; 435/325; 435/235.1; 424/9.2; 536/23.72

(57) ABSTRACT

This invention relates to nucleic acids encoding HCV/GBV-B constructs and chimeric, infectious HCV/GBV-B viruses that comprise a non-structural (NS) protein function of hepatitis C virus (HCV). The invention further relates to methods for using the nucleic acid constructs and chimeric, infectious viruses to screen for HCV inhibitors in cell culture models or in animal models of viral infection.

This application claims priority under 35 U.S.C. 119(e) from U.S. application Ser. No. 60/171,469 filed Dec. 22, 1999.

### FIELD OF THE INVENTION

This invention relates to Hepatitis C/GB viral constructs that contain a GB virus-B (GBV-B) genomic backbone and depend on a Hepatitis C virus (HCV) non-structural (NS) protein function for viability. The invention further relates to using the constructs to screen for inhibitors of HCV in cell culture systems or in animal models of viral infection.

### BACKGROUND OF THE INVENTION

HCV infection is the leading etiological agent causing non-A and non-B hepatitis. It is a compelling human medical problem with more than 170 million cases estimated globally. About 80% of patients with acute HCV infection progress to chronic hepatitis which can persist for decades. Recurrent and worsening liver inflammation occurs, and often leads to more severe disease states such as cirrhosis in 20% of patients and hepatocellular carcinomas in 1-5% patients. In the U.S., the overall prevalence of anti-HCV antibodies is about 1.8%, which corresponds to an estimated 3.9 million individuals infected by HCV. Of these seropositive individuals 74% are positive for HCV RNA, which indicates an estimated 2.7 million people in the U.S. are chronically infected.

HCV is an enveloped, positive-stranded RNA virus of the Flaviviridae family, Hepacivirus genus. It has a genome that encodes a polyprotein of about 3010 to 3033 amino acids [Choo et al., Proc. Natl. Acad. Sci. USA 88: 2451-2455 (1991); Inchauspe et al., Proc. Natl. Acad. Sci. USA 88: 10292-10296 (1991); Takamizawa et al., J. Virol. 65: 1105-113 (1991)]. The polyprotein is proteolytically processed to yield mature viral protein products as follows: NH₂-C-E1-E2-p7-NS2-NS4A-NS3-NS4B-NS5A-NS5B-cooH [Grakoui et al., J. Virol. 67:1385-95 (1993); Hijikata et al., Proc. Natl. Acad. Sci. USA 88:5547-51 (1991); Lin et al., J. Virol. 68:5063-73 (1994)].

The three structural proteins C (capsid), E1, and E2 (two envelope glycoproteins), located at the amino terminus of NS2, are cleaved by a host signal peptidase of the endoplasmic reticulum. Proteolytic processing of non-structural (NS) proteins, which provide the catalytic machinery necessary for viral replication, is carried out by viral proteases NS2-3 (catalyzes cleavage at a junction site between NS2 and NS3) and NS3 (catalyzes cleavage at junction sites between the remaining NS proteins). The cleavage between NS3 and NS4A is intramolecular (cis), whereas the protease domain and NS4A cofactor separate the remaining C-terminal NS proteins from the viral polyprotein via a trans cleavage. Since the HCV NS3 protease cleaves viral NS proteins essential for viral replication and viability, it is one target for the development of therapeutic agents against HCV. Other targets include NS3 helicase, NS4B (the function of which is yet unknown), NS5A (which may play a role in interferon resistance), and NS5B (which encodes RNA-dependent RNA polymerase function).

The development of a vaccine for HCV has been hampered by immune evasion and lack of protection against reinfection, even when using the same inoculum. The lack of a broadly effective treatment for the debilitating progression of HCV has prompted an intense effort to develop an effective small molecule inhibitor against specific HCV viral targets, such as the NS proteins. High throughput enzyme-based screening assays are being developed to screen for inhibitors that target HCV viral proteins. Several cell-based antiviral assays exist for evaluating the efficacy of potential inhibitors against the HCV protease, which employ HCV NS3 protease-dependent chimeric viruses, e.g., sindbis and poliovirus [Filocamo et al., J. Virol. 71: 1417-1427 (1997); Filocamo et al., J. Virol. 73: 561-575 (1999); Hahm et al., Virol. 226: 318-326 (1996); Cho et al., J. Virol. Methods. 65: 201-207 (1997)]. Although these chimeric viruses allow in vitro testing of cell permeation and antiviral efficacy of HCV protease inhibitors, they fail to duplicate the polyprotein processing events that naturally occur during HCV infection. In addition, useful animal models that can further test inhibitors have not been developed for these chimeric viruses.

A chimeric virus that closely mimics an HCV infection in a small primate model would prove advantageous in the development of a treatment for HCV. However, there is no reliable cell culture system or small animal model that supports HCV replication to evaluate the antiviral efficacy of HCV inhibitors. Efforts to establish a transgenic mouse model or xenografted SCID-hu mouse model have not yielded any reliable or convincing results suitable for anti-HCV drug development. The only presently accepted animal model that is permissive to HCV infection is chimpanzee, however, its limited availability, large size and high cost make it less amenable to support routine HCV drug discovery efforts or basic research. The lack of a convenient, reliable and cost-effective in vivo model that supports HCV replication or mimics HCV enzymatic functions creates an obstacle for the development of effective inhibitors.

Thus, there is a need in the art for a suitable viral and small animal model to evaluate candidate HCV inhibitor compounds. Ultimately, a model that supports both in vitro and in vivo testing would prove most beneficial for the development of HCV inhibitors. The present invention addresses these and other needs in the art.

### SUMMARY OF THE INVENTION

This invention advantageously provides HCV/GBV-B nucleic acid constructs and chimeric viruses, useful for studying HCV replication and infection and for screening of HCV inhibitors. The chimeric viruses of the invention remain fully functional and infectious. The phylogenetic proximity of GBV-B to HCV, and ability of GBV-B to infect small primates, e.g., new world monkeys, permitted the development of a unique in vivo model in which the chimeric viruses of the invention provide HCV-like infection in primate species, much smaller than a chimpanzee. The small size of these primates permits cost-effective in vivo screening for inhibitors of HCV.

Thus, it is an object of the invention to provide chimeric nucleic acid constructs encoding a GBV-B genomic backbone that has a GBV-B nucleic acid sequence encoding an NS protein, e.g., NS3, NS4A, NS4B, NS5A or NS5B, replaced by a nucleic acid sequence encoding an analogous HCV NS protein. It is another object to provide functional chimeric HCV/GBV-B viruses that comprise a nucleic acid construct of the invention.

In one embodiment, the GBV-B NS3 protease coding sequence, which encodes a protein responsible for cleaving the viral polyprotein, is replaced by a proteolytic function encoded by an HCV NS3 in a construct or virus of the invention. In this embodiment, a GBV-B NS4A cofactor coding sequence, which encodes a protein required for the enhanced function of the NS3 protease, is also replaced by a nucleic acid encoding an HCV NS4A cofactor function. Thus, in a specific embodiment, a construct or virus of the invention comprises a GBV-B genomic backbone wherein the nucleic acid sequences encoding the GBV-B NS3 protease and NS4A cofactor are replaced with nucleic acids encoding a functional HCV NS3 protease and NS4A cofactor.

In another embodiment, the GBV-B NS5B RNA-dependent RNA polymerase (RdRp) coding sequence, which encodes a protein responsible for binding viral RNA and directing RNA synthesis during replication, is replaced with an RdRp function encoded by HCV NS5B. Thus, another embodiment of the invention includes a nucleic acid construct and chimeric virus comprising a GBV-B genomic backbone wherein the nucleic acid sequence encoding a GBV-B NS5B RdRp is replaced with a nucleic acid encoding HCV RdRp function.

Other constructs and viruses comprising a GBV-B backbone and HCV NS coding sequence, e.g., NS3 helicase, will be appreciated by those having ordinary skill in the art and are described in the present application.

It is another object of the invention to provide chimeric viruses that can be propagated in vitro (in a cell culture) or in vivo (in an animal model), in which viral infectivity requires a functional HCV protein. Thus, another embodiment of the invention provides methods for propagating an infectious, chimeric HCV/GBV-B virus by culturing cells or infecting a mammal with a chimeric virus of the invention under conditions that permit production of viable viruses.

It is a further object of the invention to provide an in vitro cell culture system and an in vivo animal model for evaluating inhibitors of HCV NS proteins, such as HCV NS3 protease and NS4A cofactor and HCV NS5B RdRp. Thus, in yet another embodiment, a viral construct of the invention can be used in a method to screen for compounds that inhibit an HCV protein in vivo. Such a method evaluates viral load or disease progression, such as pathogenesis, or both, in susceptible animals infected with a chimeric virus of the invention when treated (test animal) with a candidate compound or untreated (control animal). A reduction in viral load or disease progression in the test animal relative to the control animal suggests that the compound can inhibit an HCV infection. In cell culture systems, methods are used to screen for inhibitors of HCV proteins by evaluating viral infection of the cells, such that one group of cells is cultured in the presence of a candidate compound (test cells) and a different group of cells is cultured in the absence of the candidate compound (control cells). A reduction in viral infection in test cells relative to control cells indicates that the compound inhibits an HCV protein.

The present invention meets these and other objects of the invention, as set forth in greater detail in the Detailed Description of the Invention and Examples sections, including the accompanying Figures.

### BRIEF DESCRIPTION OF THE DRAWING

FIG. 1 shows schematic drawings of GBV-B and HCV polyproteins with junction sites. FIG. 1, panel A is a full-length GBV-B genome showing the location of junction sites in the polyprotein (as numbered according to the amino acid positions in GBV-B polyprotein) that HCV NS3 protease and NS4A cofactor recognize as substrates for processing. The junction sites in the structural region have not been mapped. FIG. 1, panel B is a full-length HCV genome showing the location of junction sites in the polyprotein (as numbered according to the amino acid positions in HCV polyprotein) that GBV-B NS3 protease and NS4A cofactor recognize as substrates for processing. FIG. 1, panel C is an example of one embodiment of a chimeric HCV/GBV-B genome, wherein the NS3 protease domain of GBV-B is replaced with a functional HCV NS3 protease domain and the GBV-B NS4A cofactor is replaced with a functional HCV NS4A cofactor. The borders of NS3 and NS4A chimera were numbered according to the amino acid positions in the chimeric genome. FIG. 1, panel D shows schematic drawings of a chimeric genome consists of a functional RdRp wherein the entire GBV-B NS5B gene is replaced by an HCV RdRp.

FIG. 2 is a schematic drawing of various plasmid constructs and cofactor peptides used to test and prepare constructs of the invention recited in Example 1. The numbers above or below each construct or peptide indicate the amino acid positions corresponding to the published full-length polyprotein for each virus [Simons et al., Proc. Natl. Acad. Sci. USA 92: 3401-3405 (1995)]. The predicted molecular masses for each substrate and its cleavage products are indicated under each substrate plasmid.

FIG. 3 is an SDS-PAGE gel showing cofactor-dependent trans-cleavage activity of GBV-B NS3 protease on HCV substrates. Panel A shows cleavage of the NS4A/NS4B (Δ4A/Δ4B) substrate. Panel B shows cleavage of the NS4B/NS5A (Δ4B/Δ5A) substrate. Panel C shows cleavage of the NS5A/NS5B (Δ5A/Δ5B) substrate.

FIG. 4 is an SDS-PAGE gel showing cofactor-dependent trans-cleavage activity of GBV-B NS3 protease on GBV-B substrates. Panel A shows cleavage of the NS4A/NS4B (Δ4A/Δ4B) substrate. Panel B shows cleavage of the NS4B/NS5A (Δ4B/Δ5A) substrate. Panel C shows cleavage of the NS5A/NS5B (Δ5A/Δ5B) substrate. Each panel also shows the results of cleavage of the substrates using a mutant GBV-B NS3 (NS3S_{137A}) protease (lane 4 of each panel).

FIG. 5 is a SDS-PAGE gel showing cofactor-dependent trans-cleavage activity of HCV NS3 protease, in the presence or absence of HCV NS4A cofactor peptide (Δ4A₁₃), on GBV-B substrates. Panel A shows trans-cleavage reactions using GBV-B NS4A/4B as the substrate. Decreasing concentrations of protease (2.5, 0.25, 0.025, 0.0025 μM) were tested, indicated by a descending triangular slope. Panel B shows trans-cleavage reactions using GBV-B NS 5A/5B as the substrate.

FIG. 6 is a map of the minimum region within GBV-B NS4A that is required for cofactor activity. Sequences of the entire HCV and GBV-B NS4A proteins are aligned at the top with the minimum cofactor region (aa 22-33) for HCV NS4A underlined. A “|” represents identity, while a “:” represents similarity between the two viruses within the NS4A region. A short dash, “-” represents a gap created to maximize the homology alignment. Amino acids critical for cofactor activation of the NS3 protease are shown in bold type of a larger font; the minimum GBV-B NS4A cofactor region is also underlined (aa 22-36). Each of the peptides was tested for cofactor activity, which was graded by comparing the enhancement of the protease activity to the backgound (i.e., cleavage activity in the absence of NS4A cofactor). “+++”, 11-20 fold; “++”, 5-10 fold; “+”, 2.5-5 fold; “±”1.5-2 fold; and “−”, less than 1.5 fold over the background activity.

FIG. 7 is an SDS-PAGE gel showing that the activity of an NS3 protease is dependent upon a virus-specific NS4A cofactor. The activity of HCV or GBV-B NS3 protease was tested in the presence or absence of an HCV NS4A (Δ4A₁₃) peptide or GBV-B NS4A (Δ4A₁₇) peptide. Assays were performed using an NS4A/4B substrate from either HCV (panel A) or GBV-B (panel B).

FIG. 8 shows an SDS-PAGE gel demonstrating protease activity (panel A) and a native PAGE gel demonstrating RNA helicase activity (panel B) of a chimeric NS3 protein in comparison with native NS3. In panel A, decreasing concentrations of the GBV-B or HCV NS3 (μM) are represented by descending triangular slopes. In panel B increasing concentrations of wild type GBV-B and chimeric NS3 (pmole) are represented by ascending triangular slopes. The “Δ” indicates that a sample was boiled before loading onto the gel, and “−” represents no enzyme controls.

FIG. 9 shows expression and purification of an enzymatically active GBV-B NS5B protein. Panel A is a hydropathy profile of GBV-B NS5B. The sequence of the C-terminal hydrophobic region consisting of 19 amino acids is indicated. This region was deleted to improve solubility of the protein (GBV-B NS5B ACT19) in E.coli. Panel B shows the purification of NS5B ΔCT19 expressed in E. coli. Lane 1, uninduced total cell lysate (UN); lane 2, total cell lysate after induction (IND) with 0.2 mM IPTG; lane 3, soluble fraction from the induced lysate; and lane 4, eluate from the Ni-NTA affinity column. The upper panel was stained with coomassie blue and the lower panel is a Western blot of the same samples using an anti-penta-histidine monoclonal antibody (Qiagen). Panel C shows the RdRp activity of GBV-B NS5B ΔCT19. Lane 1, RNA size marker; lane 2, input RNA template end-labeled by T4 polynucleotide kinase and γ-³³P-ATP. Lanes 3 to 8 are RdRp reactions in the presence of increasing concentrations of ZnCl, (0, 1, 2.5, 5, 7.5, 10 μM), respectively.

FIG. 10 shows the effects of time (panel A), temperature (panel B), pH (panel C), and glycerol (panel D) on polymerase activity of GBV-B NS5B ΔCT19.

FIG. 11 describes effects of monovalent and divalent cations for GBV-B NS5B activity. Panel A reports the effects of monovalent cations, KCl and NaCl, at concentrations between 0 and 200 mM. Panel B reports the effects of divalent cations, MnCl₂ and MgCl₂, at concentrations between 0 and 20 mM.

FIG. 12 shows the template preference for RNA binding and RNA synthesis. Panel A reports the RNA-binding activity of GBV-B NS5B ΔCT19 to an end-labeled synthetic RNA probe (1.0 pmole). Lane 1, no enzyme was added; lane 2, 100 ng of enzyme; lane 3, treatment with proteinase K (2.8 mg per ml) at 37° C. for 15 minutes; Lanes 5 to 8, 10 to 13, 15 to 18, and 20 to 23 are competition experiments with increasing concentrations of poly (U), poly (A), poly (C) and poly (G), respectively. The amounts of the competing RNA homopolymers are 0.1 (lanes 5, 10, 15 and 20), 1.0 (lanes 6, 11, 16 and 21), 10 (lanes 7, 12, 17 and 22) and 100 pmoles (lanes 8, 13, 18 and 23). Panel B shows the activity of RNA homopolymers as template for GBV-B NS5B by SPA assay.

### DETAILED DESCRIPTION OF THE INVENTION

This invention provides chimeric HCV/GBV-B nucleic acids and infectious viruses that comprise a GBV-B genomic backbone, and which require the functional activity of at least one HCV protein for infectivity and viability. The chimeric viruses can be used in cell culture or in vivo, such as for studying viral activity and for evaluating inhibitor compounds of HCV which may specifically target an HCV protein function. Conventional in vitro, enzyme-based [Kwong and Risano, 1999, Development of a hepatitis C virus RNA helicase high throughput assay, p. 97-116. In Antiviral methods and protocols, vol. 24. D. Kinchington and R. F. Schinazi (ed.) Humana Press Inc., Totowa, N.J.; Ferrari et al., J. Virol. 73:1649-1654 (1999);Taremi et al., Protein Sci. 7:2143-2149 (1998)]and cell culture assays [Burleson et al., Virology: a laboratory manual. Academic Press, New York (1992)] and animal studies [Bukh et al., Virol. 262: 470-478. (1999)] can be used for this purpose, and are well-known in the art. In a preferred embodiment, a GBV-B viral construct of the invention infects tamarin monkeys (Saguinus species), since these animals provide a convenient model for screening (discussed below). Preferably, a GBV-B clone that is not infectious in humans is selected so that all work can be performed with no issues of biohazards.

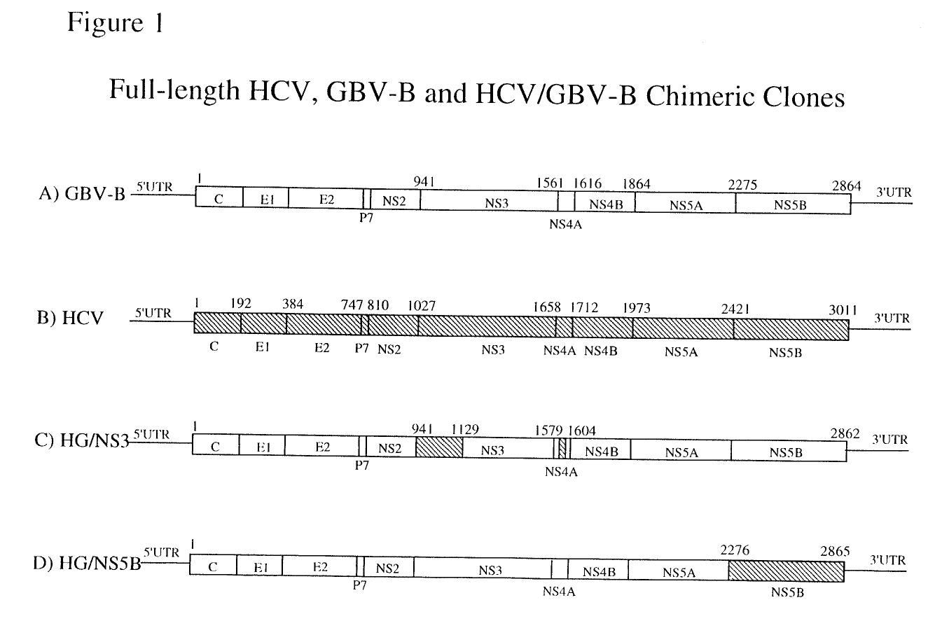

The genome of GBV-B contains an open reading frame (ORF) encoding a single polyprotein of approximately 2860 amino acids [Muerhoff et al., J. Virol. 69: 5621-5630 (1995); Ohba et al., FEBS Lett. 378: 232-234 (1996)]. See FIG. 1. Among all animal viruses, GBV-B (associated with GB agent hepatitis) shares the closest homology with HCV [Muerhoff, 1995, supra; Simons, 1995, supra] based on phylogenetic analysis and genome organization, and is similar to HCV in the following ways. It is an enveloped, positive-stranded RNA virus and shares similar IRES structure/function and polyprotein organization. The GBV-B polyprotein is processed into several structural (C, E1, E2 and p7) and nonstructural (NS2, NS3, NS4A, NS4B, NS5A and NS5B) proteins by either host or virally encoded proteases [Muerhoff et al., 1995, supra]. GBV-B is hepatotropic, causing liver disease in susceptible primates. Based on its similarites to HCV, GBV-B has provided the ability to develop unique surrogate models of the invention for evaluating anti-HCV inhibitors.

A significant difference between GBV-B and HCV is the ability of GBV-B to infect small primates, e.g., new world monkeys such as tamarins. Despite the remarkable similarities, HCV does not infect small primates, which is why the chimpanzee has been the conventional animal model. However, there are several disadvantages to using a chimpanzee model to study HCV. At minimum, it generally takes about 6 to 8 weeks for hepatitis to develop in chimpanzees infected with HCV, whereas the disease may take as little as one week to develop in a small primate. This main difference in time to establish infection, in light of the similar characteristics between HCV and GBV-B in sequence homology, liver tropism, and polyprotein organization, provides a more efficient surrogate model for studying HCV infection.

Another advantage of the use of tamarins as an animal model is its small size (approximately 500 grams in weight). This allows rapid screening of inhibitors at an early discovery stage before scaling up the production of any compound of interest.

Thus, there are numerous advantages to using chimeric viruses comprising a GBV-B backbone for studying HCV infection and inhibitors, including a smaller and more cost-effective animal model, similar host and tissue tropism, and faster disease progression to allow efficient in vivo studies of HCV inhibitors.

### Definitions

A “construct” is a chimeric virus or a nucleic acid encoding the genome of a chimeric virus, such as positive viral genomic RNA or a DNA that can be transcribed to produce viral genomic RNA. The term “chimeric” is used herein in its usual sense and refers to a construct resulting from the combination of genes from two different sources in which the different parts of the chimera function together. The genes are fused, where necessary in-frame, in a single genetic construct. The use of “chimeric” or “construct” with “HCV/GBV-B” or “virus” generally refers to a GBV-B genome in which a gene encoding an HCV NS protein function is inserted. Preferably, the GBV-B genome is modified, e.g., by deletion of a GBV-B gene which is analogous to the inserted HCV NS gene, so that a chimeric virus requires activity of the HCV NS protein for viability and infectivity.

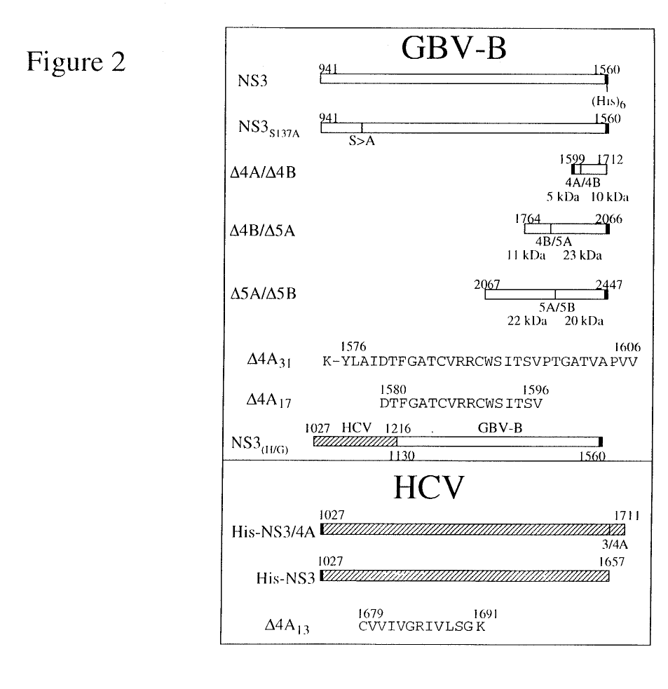

The terms “junction site”, “junction”, or “substrate” are used interchangeably to refer to an amino acid sequence joining two different proteins of a viral polyprotein, that is recognized and proteolytically cleaved, e.g., by the NS3 protease domain. The location and sequences of the various HCV and GBV-B NS junction sites are known (see FIGS. 1 and 2), including the NS3/NS4A, NS4A/NS4B, NS4B/NS5A, and NS5A/NS5B junction sites.

A “GBV-B” is a viral species of the Flaviviridae family that infects mammals, including small primates such as new world monkeys, e.g., tamarins (Saguinus species). An example of a GBV-B nucleic acid clone and polyprotein is represented by SEQ ID NO: 1 and SEQ ID NO: 2, respectively, and shown schematically in FIG. 1. Modifications to a GBV-B genome to prepare constructs of the invention are within the ordinary skill of the art and further discussed infra.

The term “infectious” refers to the ability of a virus to replicate in a cell and produce viral particles that are capable of infection. It can also refer to detection of expressed proteins from a chimeric viral genome, using conventional techniques known in the art, which indicates that the viral construct is infectious. An infection in vivo is development of either an acute or chronic viral infection, which may include either overt pathology or merely replication and propagation of the virus in an infected animal. Plaque formation (also termed “cytopathic effect” or “cell lysis”) of cells in culture can be used as evidence that a viral construct is infectious. However, the absence of plaque formation is not conclusive that viral infection of a cell has not occurred, since cells infected with a noncytopathic virus can appear indistinguishable from uninfected cells. Other means for detecting viral infection are known in the art, and include immunostaining and RT-PCR methods to detect insidious noncytopathic viral infection.

The term “viral load” refers to a quantitative amount of virus in an animal, detectable using ordinary means known in the art. An animal that permits a viral infection and replication is referred to as “viremic”.

“Disease progression” refers to the course of disease or symptoms of an infected animal, and may include acute or chronic disease symptoms. “Pathogenesis” refers to the pathogenic effects of viral infection. In the present invention, disease progression and pathogenesis refer to events typically associated with an HCV or GBV-B infection, including hepatitis. “Hepatitis” is used in its ordinary meaning to refer to the pathology and symptomology associated with viral infection by HCV or GBV-B. Typically, viral hepatitis is associated with liver injuries marked by inflammation and elevated levels of liver-specific enzymes in the blood, e.g., ALT (alanine aminotransferase), AST (aspartate aminotransferase), γ-GGT (γ-glutamyl-transpeptidase), ICD (isocitrate dehydrogenase). The more severe and later forms of chronic hepatitis include cirrhosis and hepatocellular carcinoma. The manifestation of hepatitis, as well as disease progression and pathogenesis, in an animal or human can be determined by one having ordinary skill in the art.

Various aspects of the invention are disclosed in greater detail in the following sections related to viral constructs and NS proteins, strategies for preparing and propagating chimeric viruses, and screening assays using chimeric viruses.

### Molecular Biological Techniques

In accordance with the present invention, there may be employed conventional molecular biology, microbiology, or recombinant DNA techniques within the ordinary skill of the art to prepare of viral constructs of the invention. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis [M. J. Gait ed. (1984)]; Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; A Practical Guide To Molecular Cloning [B. Perbal (1984)]; Current Protocols in Molecular Biology, John Wiley & Sons, Inc. [F. M. Ausubel et al. (eds.) (1994)]; [Burleson, 1992. Virology: A laboratory manual. Academic Press, New York].

As used herein, the abbreviations “nt” and “aa” refer to “nucleotide(s)” and “amino acid(s)”, respectively.

A “nucleic acid molecule” refers to the phosphate diester polymeric form of ribonucleosides (adenosine, guanosine, uridine or cytidine; “RNA molecules”), or deoxyribonucleosides (deoxyadenosine, deoxyguanosine, deoxythymidine, or deoxycytidine; “DNA molecules”), in either a single stranded or a double-stranded helix form. Double stranded DNA-DNA, DNA-RNA and RNA-RNA helices are contemplated. The term nucleic acid molecule, and in particular DNA or RNA molecule, refers only to the primary and secondary structure of the molecule, and does not limit it to any particular tertiary forms. The structure of a particular nucleic acid molecule, sequence or region may be described herein according to the normal convention of providing a sequence in the 5′ to 3′ direction. A “recombinant DNA molecule” is a DNA molecule that has undergone a molecular biological manipulation.

The term “gene” means a DNA sequence that codes for or corresponds to a particular sequence of amino acids which comprise all or a portion of one or more proteins or enzymes. Preferably, if a gene encodes only a portion or fragment of a protein or enzyme, then it encodes a functional portion (e.g., a domain) that has an activity present in the full length protein or enzyme. For example, a viral gene encoding an HCV NS4A cofactor may encode the full length protein which typically consists of about 54 amino acids or it may encode a functional domain thereof, which may consist of only 13 amino acids (e.g., aa 22-34).

Unless otherwise noted, the term “genome” refers to a GBV-B genome which is modified by insertion or substitution of an HCV NS gene sequence and removal of an analogous GBV-B gene sequence (in relation to the HCV sequence) in constructs of the invention. The genome of a virus may be referred to as the “backbone” (e.g., genomic backbone) such as when referring to a chimeric. Although a chimeric virus of the invention will consist mostly of a GBV-B genome, properties of HCV are preserved in the chimeric virus, including infectivity, tissue tropism, and life cycle, due to the close phylogenetic proximity of GBV-B with HCV and high identity and similarity of the polyprotein at the amino acid level (approximately 37% identity and 52% similarity).

The term “homologous” describes a group of genes, proteins, nucleic acids or amino acid sequences, or the functional property of a protein, each of which are derived from the same virus. For example, HCV NS3 is homologous in reference to NS4A cofactor of HCV. The term “heterologous” describes a group of genes, proteins, nucleic acids or amino acid sequences, or the functional property of a protein, which are different in relation to a particular gene, protein, nucleic acid, amino acid sequence, or functional property of a protein and which are derived from different viruses. Thus, for example, HCV NS3 can process heterologous junction sites in substrates of GBV-B origin. The term “analogous” is used to describe the same gene, protein, nucleic acid, amino acid sequence, or functional property of a protein, which are derived from different viruses. For example, a GBV-B NS3 protease is analogous to HCV NS3 protease.

A “sequence-conservative variant” of a gene contains a change of one or more nucleotides in a given codon position and results in no alteration in the amino acid encoded at that position. A “function-conservative variant” has a change to one or more nucleotides which causes an alteration in an amino acid residue in the protein or enzyme, including, but not limited to, replacement of an amino acid with one having similar properties (such as, for example, polarity, hydrogen bonding potential, acidic, basic, hydrophobic, aromatic, and the like). The resulting amino acid in a function-conservative variant does not alter the overall conformation or function of the polypeptide.

A “coding sequence” or a sequence “encoding” an expression product, e.g., RNA, polypeptide, protein, or enzyme, is a nucleotide sequence that, when expressed, results in the production of that expression product. In the present invention, translation of a coding sequence, e.g., RNA, initially yields a polyprotein, which is cleaved during co- or post-translational processing, particularly by a protein having functional NS3 protease activity, to yield functional viral proteins.

A coding sequence is “under the control” or “operatively associated with” transcriptional and translational control sequences in a cell when an RNA polymerase transcribes the coding sequence into mRNA, which can then be translated into a protein encoded by the coding sequence.

The terms “express” and “expression” mean allowing or causing the information in a gene or DNA sequence to become manifest, e.g., producing a protein by activating the cellular functions involved in transcription and translation of a corresponding gene or DNA sequence. A DNA sequence can be expressed using in vitro translation assays or in or by a cell to form an “expression product” such as an MRNA or a protein. The expression product too, e.g. the resulting protein, may also be referred to as “expressed”.

A “domain” typically refers to a part, portion or segment of a protein or a peptide that has a particular property, e.g., a binding or catalytic activity. A peptide may comprise an entire domain. Specific properties of domains of peptides or proteins expressed by a construct of the invention are described in detail infra, when a particular domain is discussed.

A “host cell” is any cell that is capable of infection by or propagation of a chimeric viral construct of the invention. Host cells can further be used for screening or other assays, as described infra.

The term “transfection” means introduction of a foreign nucleic acid into a mammalian cell and includes introduction of a whole virus genome which contains the nucleic acid of interest. The introduced gene or sequence may also be called a “cloned”, “foreign” or “analogous” gene or sequence, and may include regulatory or control sequences, such as start, stop, promoter, signal, secretion, or other sequences used by a cell's genetic machinery. A host cell that receives and expresses introduced DNA or RNA has been “transformed” and is a “transformant”.

### Preparation of HCV/GB V-B Constructs

A construct of the invention can be prepared from GBV-B and HCV genomic clones using conventional molecular biology and virology techniques. FIG. 1 shows examples of the arrangement of a GBV-B (SEQ ID NO: 1; nt and SEQ ID NO: 2; aa) and HCV (SEQ ID NO: 3; nt and SEQ ID NO: 4; aa) genome. It is noted that HCV strains exemplified herein include HCV-1a(H) (see Inchauspe, 1991, supra) and HCV-1b(BK) (discussed in Ferrari, 1999, supra) however other HCV strains can be used to prepare constructs of the invention.

Several conventional methods can be used to prepare GBV-B and HCV clones. For example, viral RNA can be extracted from infected serum using conventional RNA extraction methods. A viral cDNA is prepared from the extracted RNA by RT-PCR using primer sequences based on the known genomic sequence of GBV-B or HCV, for example, see Simons et al., Proc. Natl. Acad. Sci USA 92:3401-3405 (1995); Kolykhalov et al., Science 277: 570-574 (1997); Yanagi et al., Proc. Natl. Acad. Sci. U S A. 94:8738-8743 (1997); and Hong et al., Virol. 256: 36-44 (1999). The PCR product is purified and cloned into a suitable vector for further manipulation. A specific example of methods for preparing GBV-B clones from the serum of infected tamarins is found in Bukh et al., supra. Specific methods for preparing HCV clones are described in Hong et al., supra.

A construct of the invention contains at least one nucleic acid sequence from HCV that encodes a functional activity of the analogous GBV-B protein. There are several possible NS genes from a GBV-B clone that can be substituted with an analogous HCV coding sequence, e.g., NS3 and NS4A, NS4B, NS5A, and NS5B, to prepare a construct of the invention, discussed infra. In any construct of the invention, a modification to an HCV gene which does not significantly impair the functional activity of the expressed protein can be made, i.e., the modification does not prevent viral infection or replication. For example, an HCV nucleic acid for insertion into a GBV-B genome may encode a full-length protein, a truncated form, or a variant that has an amino acid substitution, provided that a chimeric virus comprising the inserted nucleic acid depends on the functional activity of the HCV protein for viability and infectivity. A discussion and examples of modifications to HCV genes are found below where particular HCV NS genes are described.

It is preferred that in any construct of the invention, the GBV-B gene that is substituted by an analogous HCV gene is entirely deleted from the GBV-B genome. This ensures that the infectivity and viability of a viral construct is dependent upon the inserted or replaced HCV nucleic acid sequence encoding the desired function, and not on any remaining sequence encoding a homologous GBV-B protein.

Conventional molecular biology techniques, e.g., PCR amplification and standard cloning techniques, can be used to prepare chimeric viruses of the invention. For example, a restriction site that is specifically recognized by a restriction enzyme can be engineered at any location within a GBV-B genomic clone, e.g., at junction sites located adjacent to a coding region for a GBV-B gene that is desired to be replaced. To accomplish this, PCR can be used to generate GBV-B clones that have two (preferably different) engineered restriction enzyme cleavage sites adjacent to the junction sites, by using primers homologous to segments of the GBV-B genome, which segments comprise the adjacent junction sites and the engineered cleavage sites. The resulting, amplified GBV-B clone will have two engineered restriction enzyme cleavage sites at junction sites adjacent to the GBV-B gene to be replaced. This process can be adapted to engineer the same restriction enzyme cleavage sites at the ends of a desired HCV coding sequence from an HCV genomic clone that will replace the analogous gene in the GBV-B clone. Digesting the GBV-B clone and the HCV clone with the appropriate restriction enzymes allows excision of the genes of interest from the GBV-B and HCV clones. The HCV coding sequence is isolated, and using conventional DNA ligation methods, it is inserted at the junction sites of the GBV-B clone in the proper orientation to yield an HCV/GBV-B construct. It shall be appreciated that various other well known molecular cloning techniques and strategies may be employed by those having ordinary skill in the art to prepare a construct of the invention.

Other methods, such as PCR-based molecular techniques can be used to create chimeric constructs without having to rely on restriction enzymes. For example, “Splicing Overlap Extension PCR” (SOE-PCR) or “Bridging Overlap Extension PCR” is frequently used to join several smaller PCR fragments with overlapping ends through a second round PCR [see, e.g., Mehta and Singh, Biotechniques 26: 1082-86 (1999); Horton et al., Gene 77:61-68 (1989); Horton, Protein Sci. 8:1332-1341 (1995)].

### NS3 Protease Constructs

In one embodiment, the invention is a construct in which a GBV-B NS3 protease coding sequence is replaced by a protease domain of HCV NS3. In any NS3 protease construct, a GBV-B NS4A cofactor is also substituted by an HCV NS4A cofactor, in order to provide fully functional HCV protease activity. Thus, in an NS3 protease construct, the GBV-B NS3 protease and NS4A cofactor, which function together to cleave the polyprotein junctions, e.g., NS3-NS4A, NS4A-NS4B, NS4B-NS5A, and NS5A-NS5B, are replaced by HCV NS3 protease and HCV NS4A cofactor functions.

The NS3 gene encodes two enzymatic activities, including a proteolytic function located at the N-terminus (protease domain) and a helicase/ATPase function at the C-terminus (helicase domain, described further infra). An HCV NS3 protease domain is 190 amino acid residues of the HCV polyprotein corresponding to aa 1027 to 1216 of SEQ ID NO: 4. The analogous protease domain encoded by GBV-B NS3 is 189 amino acids, corresponding to aa 941 to 1129 of SEQ ID NO:2.

A short and linear stretch of amino acids in NS3 (aa 1210 to 1230 in HCV and aa 1125 to 1135 in GBV-B) form a peptide tether which joins the protease and helicase domain to make a full-length, bifunctional NS3 protein. However, since the protease and helicase domains are separated by a short stretch of amino acids and have minimal interaction, each can be manipulated without compromising the activity of the other domain.

Proper functional activity of an HCV NS3 serine protease relies on an active site that comprises a His-Asp-Ser catalytic triad located at amino acids 57, 81 and 139, respectively. Therefore, the sequence must be present in an NS3 protease construct of the invention to provide minimal protease activity. Mutation of the Ser residue abolishes the ability to cleave at NS3/4A, NS4A/4B, NS4B/5A, and NS5A/5B junctions.

An HCV NS3 protease used in a construct of the invention is preferably an entire HCV NS3 protease domain containing amino acids 1 to 190 of an NS3 protein, but it can be truncated (e.g., to contain at least amino acids 1 to 160) or mutated with one or more point mutations, or a function- or sequence-conservative variant that has a divergent sequence either at the nucleotide or amino acid level. Any modification to an HCV protease domain used in a construct of the invention is permissible, as long as the functional activity of the protease is preserved, i.e., at least a functional, proteolytically active form or domain of an HCV NS3 protein. An HCV protease used in a construct of the invention can process a GBV-B polyprotein to yield NS proteins, and ultimately chimeric viruses that are infectious.

Various U.S. Patents disclose examples of functional HCV proteins, inter alia, including U.S. Pat. No. 5,371,017 (isolated polynucleotide which encodes only the HCV protease or an active HCV protease analog), U.S. Pat. Nos. 5,585,258 and 5,712,145 (compositions comprising an HCV NS3 protease domain or truncation analog).

An example of an HCV/GBV-B NS3 chimeric protein of the invention, which can be used in a viral construct of the invention, is provided by SEQ ID NO: 7. This sequence represents a chimeric NS3 protein wherein the C-terminal protease region from GBV-B, corresponding to aa 941 to 1129, is replaced with the C-terminal protease region from HCV, corresponding to aa 1027 to 1215, (i.e., aa 1 to 189 of SEQ ID NO: 7).

An example of an NS3 protease viral construct of the invention is provided by SEQ ID NO: 5, which also has an HCV NS4B cofactor, that replaces the analogous coding regions of GBV-B. In this particular construct, an NS3 protease domain corresponding to aa 1031 to 1215 of HCV (SEQ ID NO: 4) replaces an NS3 protease domain corresponding to aa 941 to 1129 of GBV-B (SEQ ID NO: 2). In addition, this construct has NS4A cofactor sequences from HCV, as described below. It is noted that in the construct set forth in SEQ ID NO: 5 HCV sequences were obtained from HCV-1a(H), however other HCV strains can be used without departing from the spirit of the invention.

Conventional assays known in the art can be used to test the functional activity of an HCV NS3 protease construct of the invention. For example, contacting the protein encoded by a chimeric protease NS3 gene with a GBV-B polyprotein and observing cleavage at the junction sites can be used to demonstrate the functional activity of the protease. Such an assay is described in a specific embodiment of the invention, described in Example 1, infra.

### NS4A Cofactor

Experiments using transient expression of various forms of HCV NS polyproteins in mammalian cells have established that an NS3 serine protease is necessary, but not sufficient, for proteolytic processing at viral polyprotein junction sites [Butkiewicz, Virology 225: 328-338 (1996); Lin et al., J. Virol. 69: 4373-4380 (1995); Tomei et al., J. Gen. Virol. 77: 1065-1070 (1996)]. It has now been discovered that, surprisingly, viral NS3 protease activity is further dependent on an interaction with a viral-specific NS4A cofactor for complete processing of the polyprotein junction sites. Therefore, a construct of the invention that comprises an HCV NS3 protease requires an NS4A cofactor that is derived from HCV, in order to properly process the GBV-B polyprotein.

A full length HCV NS4A cofactor comprises 54 amino acid residues as shown in FIG. 1B, which corresponds to amino acids 1658 to 1711. An analogous GBV-B NS4A domain is also shown in FIG. 1A. A truncated, mutated (having one or more point mutations), or a function- or sequence-conservative variant (having a divergent sequence either at the nucleotide or amino acid level) of HCV NS4A can also be employed, provided the functional activity is presented.

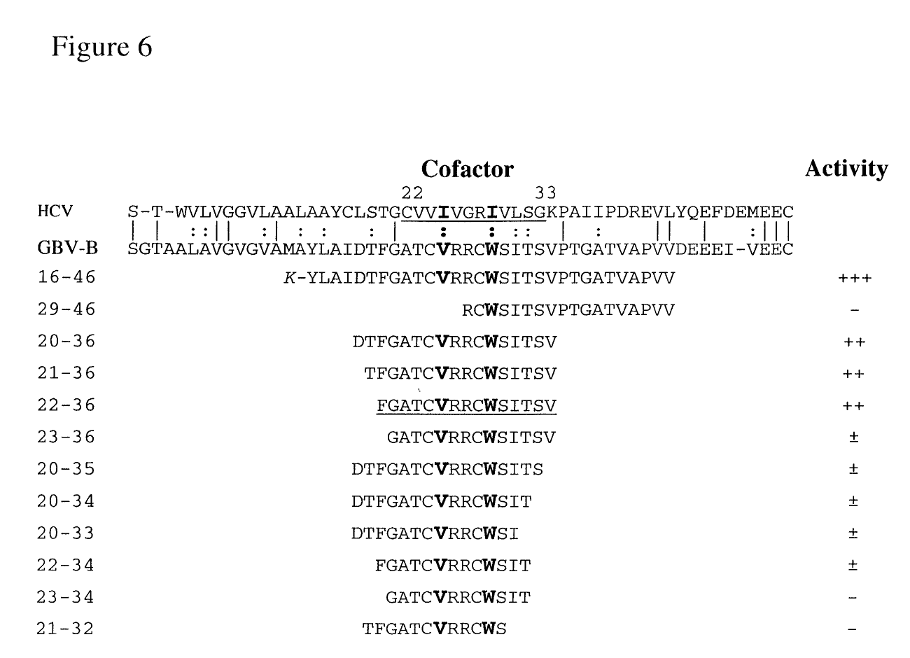

Thus, the minimum sequence of an HCV NS4A protein necessary for NS3-mediated proteolytic activity is a 13 residue central domain spanning residues 22-34 (FIG. 6). It is preferred that no substitutions are made to V₂₃, V₂₄, V₂₆ G₂₇ and I₃₁, as any one of these substitutions may be detrimental to NS4A function, and consequently to NS3 protease activity. It is most preferred, in order to maintain minimal levels of NS3 protease activity, that there are no substitutions to I₂₅ and I₂₉, as either substitution may abolish activity of NS4A. Modifications and substitutions to the NS4A coding region can be made and tested by one having ordinary skill in the art using known molecular biology techniques. Conventional assays for detecting NS3 protease activity can be employed for this purpose, as discussed above.

An example of an HCV/GBV-B NS4A chimeric protein of the invention, which can be used in a viral construct of the invention, is provided by SEQ ID NO: 8. This sequence represents a chimeric NS4A protein wherein the residues in the central domain of the NS4A cofactor from GBV-B, corresponding to aa 1579 to 1596, is replaced with a 16 residue central domain from HCV NS4A, corresponding to aa 1676 to 1691 (aa 19 to 34 of SEQ ID NO: 8). As mentioned above, an example of a viral construct of the invention which comprises an HCV NS4A cofactor that replaces the analogous protein of GBV-B is provided by SEQ ID NO: 5. In this particular construct, the 16 residue central domain from HCV NS4A replaces an analogous NS4B region of the GBV-B genome, as described supra.

### HCV NS3 Helicase Constructs

In another embodiment, the invention is a construct in which the nucleotide coding sequence of GBV-B NS3 helicase is replaced by a coding sequence for a helicase domain of HCV to provide a chimeric HCV/GBV-B that has functional HCV helicase activity. Thus, in this particular construct of the invention, the NS3 protease is of GBV-B origin while the helicase domain is from HCV.

An HCV NS3 helicase/ATPase domain is 441 amino acids, corresponding to amino acids 1217 to 1657. The analogous NS3 helicase/ATPase domain in GBV-B is 431 amino acids, corresponding to amino acids 1130 to 1560.

An HCV NS3 helicase used in a construct of the invention is preferably an HCV NS3 helicase/ATPase domain containing amino acids 1217 to 1657 of HCV polyprotein, but it can be truncated (e.g., to contain at least amino acids 1225 to 1600), mutated (with one or more point mutations), or a function- or sequence-conservative variant (having a divergent sequence either at the nucleotide or amino acid level). Any modification to an HCV helicase domain used in a construct of the invention is permissible, as long as the helicase/ATPase functional activity is preserved, i.e., at least a functional and active form or domain of an HCV NS3 protein. An HCV helicase used in a construct of the invention can be characterized using conventional assays known in the art [see, e.g., Kwong and Risano, supra; Lee et al., J. Biol. Chem. 267: 4398-4407 (1992)].

Conventional assays known in the art can be used to test the functional activity of an HCV NS3 helicase construct of the invention. For example, contacting the protein encoded by a chimeric helicase NS3 gene with a double-stranded GBV-B or other double-stranded RNA to unwind the two strands. The unwinding event can be observed on a gel that separates single-stranded RNA from double-stranded RNA using conventional assays known in the art for testing and observing RNA helicase/ATPase activity (see, e.g, [Kwong and Risano, 1999, supra; Lee, 1992, supra]. Such assays are well known in the art, an example of which is described in a specific embodiment of the invention, described in Example 1, infra.

### HCV NS5B RNA-Dependent RNA Polymerase Constructs

In another embodiment of the invention, a construct is provided in which a GBV-B NS5B gene encoding an RNA-dependent RNA polymerase (RdRp) is replaced by a coding sequence for an RdRp domain of HCV NS5B such as, e.g., the construct of SEQ ID NO: 6. It is noted that the HCV sequence of this particular construct was obtained from HCV-1b(BK), however other HCV strains can be used without departing from the spirit of the invention.

An HCV NS5B RdRp domain is 591 amino acids, corresponding to amino acids 2421 to 3011 of SEQ ID NO: 4. The analogous NS5B domain in GBV-B is 590 amino acids, corresponding to amino acids 2275 to 2864 of SEQ ID NO: 2. In an NS5B construct, the replacement of a GBV-B RdRp with HCV RdRp activity provides a chimeric HCV/GBV-B that depends on a fully functional HCV RdRp for viral replication and, thus, for viability. Thus, a chimeric NS5B construct comprises, at least, a functionally active HCV NS5B RdRp domain.

The NS5B gene encodes a catalytic subunit protein that has RNA-dependent RNA polymerase activity, i.e., it works in concert with other viral and host proteins to catalyze the replication of the viral RNA genome. The RdRp activity of NS5B also includes the ability to catalyze nucleotidyl transfer by extending the 3′ hydroxyl of a template (“copyback”) or of an RNA or DNA primer [Behrens et al., EMBO J. 15:12-22 (1996); Lohmann et al., J. Virol. 71:8416-8428 (1997)].

An NS5B protein from HCV and GBV-B comprises at least three structural subdomains of similar sizes which are characterized based on a right-hand orientation: a central palm domain, a finger domain located N-terminal relative to the palm domain, and a thumb domain located C-terminal relative to the palm domain. The central palm domain comprises at least five polymerase motifs (A to E), which constitute the catalytic center of the RdRp activity.

An HCV RdRp used in a construct of the invention is preferably a full-length HCV NS5B RdRp (e.g., FIG. 1D), but can be truncated, mutated (having one or more point mutations), or a function- or sequence-conservative variant that has a divergent sequence either at the nucleotide or amino acid level, provided a modification does not abolish the infectivity of the chimeric virus. It may be desirable to prepare a chimeric NS5B gene for insertion into a construct of the invention, i.e., the resulting NS5B gene encodes domains from both HCV and GBV-B. In such chimeric NS5B genes, it is preferred that a palm domain from HCV is employed. Thus, several chimeric NS5B genes may be prepared to replace the native NS5B gene of GBV-B, including a GBV-B finger-HCV palm-GBV-B thumb NS5B, a GBV-B finger-HCV palm-HCV thumb NS5B, and an HCV finger-HCV Palm-GBV-B thumb NS5B. Modifications to NS5B domains and preparation of chimeric NS5B genes are within the ordinary skill of the art, and the resulting activity of NS5B can be tested using standard assays for viral RdRp activity (e.g., Ferrari et al., J. Virol. 73:1649-1654 (1999) and Example 2, infra). In any NS5B construct of the invention, whether a full-length NS5B protein is employed, a fragment thereof, or a chimeric NS5B, the substituted NS5B gene has the functional activity of a viral RdRp, i.e., ability to bind and replicate viral RNA for producing infectious viruses.

### Propagation of Viruses

GBV-B clones and chimeric viruses of the invention can be propagated in bacterial hosts using standard molecular biology techniques. See, e.g., Bukh et al., supra. General methods for propagating viruses in vivo, e.g., by serial passages of infected serum in naive animals via intravenous injection of a transmissible amount of a chimeric virus, are also generally known [see, e.g., Hong et al., supra]. Plasmophoresis techniques can be performed to obtain large quantities of virus-containing serum pools.

### Screening

The chimeric viruses and constructs of the invention are useful in secondary validation assays for compounds selected for the ability to inhibit HCV NS proteins using conventional primary screens known in the art, e.g., [Kwong and Risano, supra; Ferrari et al., supra; Taremi et al., supra]. Secondary screens can be divided into in vitro cell culture assays, using host cells, and in vivo animal models of viral infection and pathogenicity. The selection of host cells and animals for these assays will depend on the tropism of GBV-B, since it is the viral backbone of the constructs of the invention, which selection can be made by one having ordinary skill in the art.

Any discovery process can be used to evaluate a compound for use in a secondary screen of the invention. Primary screens, such as enzymatic studies can be designed according to conventional protocols, employing an HCV protein that is prepared for use with a construct of the invention. For example, as described in PCT/US98/24528, a covalent NS4A-NS3 complex, along with known synthetic substrates, can be used to develop high throughput assays. These assays can then be used to screen for compounds that inhibit the proteolytic activity of the NS3 protease. This is carried out by developing techniques for determining whether or not a compound will inhibit the covalent NS4A-NS3 complexes of the invention from cleaving the viral substrates. An example of such a high throughput assay is the scintillation proximity assay (SPA). SPA technology involves the use of beads embedded with scintillant. The beads are coated with acceptor molecules which can capture either substrate or product of an enzymatic reaction. U.S. Pat. No. 5,597,691 describes similar assays for identifying HCV NS3 inhibitors. Ferrari et al. [J. Virol. 73: 1649-1654 (1999)] describe primary screen assays that can be used for identifying HCV NS5B inhibitors. Kwong and Risano (supra) report primary high throughput assays for identifying HCV NS3 RNA helicase inhibitors.

### Cell Culture Assays

Cell culture assays can be performed by either transfecting cultured host cells with transcribed viral genomic RNA encoding a chimeric virus of the invention or infecting host cells with virus. Preferably, mammalian or tamarin host cells (e.g., hepatocytes) are used, particularly as these cells are infected by GBV-B.

To perform these assays, cultured host cells are infected with a chimeric virus of the invention, according to known methods for infecting cells with viral RNA (e.g., Xu et al., J. Virol. 71: 5312-5322 (1997); Mendez et al., 72: 4737-4745 (1998)). A group of infected cells are treated with the test compound. Negative control cells are untreated (control group) or treated with a sham test compound, such as buffer or buffer with a compound that does not inhibit the activity of the substituted HCV protein. Other positive control cells may be treated with a compound known to inhibit HCV activity.

The cells are maintained in culture under conditions that permit viral replication which may form plaque or develop cytopathic effect (CPE). The test and control cultures are then evaluated for inhibition of the chimeric virus. If cells treated with a test compound nevertheless allow viral replication and develop plaque or CPE, the compound does not effectively inhibit activity. However, if cells treated with a test compound stop viral replication and reduce plaque formation or CPE (and the negative controls do not), then the compound is effective at inhibiting the substituted HCV protein.

### In vivo Animal Models

In one embodiment of the invention, a chimeric GBV-B having tropism for small mammals is employed for studying inhibitors of HCV in vivo. Preferably, the animal is a new world monkey, such as a tamarin (Saguinus species). This will permit large scale testing for dose response under various conditions, toxicology, and pharmacokinetics, since groups of these animals are available and easier to work with than the conventional chimpanzee model. Methods for infecting animals with viruses are generally known in the art and can be found in Vitral et al., Braz. J. Med. Biol. Res. 31: 1035-1048 (1998) and Schaluder et al., J. Med. Virol. 46: 81-90 (1995). An example protocol for infecting tamarins with GBV-B is found in Bukh et al., supra.

Infectivity can be evaluated, such as by detecting virus (e.g., determining viral load) in the blood or tissue of an infected animal or by observing disease progression in the animal. Virus (viral load) can be detected by the presence of viral nucleic acids, e.g., detected by PCR or RT-PCR or direct hybridization techniques. It can also be detected, if present in sufficient amount, by the presence of viral proteins, e.g., detected by immunoassay or conventional biochemical techniques. In another alternative, extracts from tissue samples from the animal can be used to infect host cells in culture; the presence of virus is detected by development of CPE.

Pathogenicity can be evaluated by gross examination of symptomology of the animal, such as elevation of ALT, AST, etc., which are indicative of hepatitis, and histopathology evaluation of liver biopsies. Evaluation can be part of an acute condition, i.e., arising and resolving over a short period, e.g. 10 to 16 weeks, or a chronic condition, i.e., persisting for a long period of time, e.g., six months or longer.

Genetic stability of a chimeric virus in vivo can also be determined by isolating viral nucleic acid from the blood or tissue of an infected animal and directly sequencing regions of the chimeric viral genome that comprise the coding sequence for the HCV protein. All animal-derived virus growth assays include a side-by-side control consisting of a GBV-B genomic backbone derived from the infectious clone used to construct the chimeric clone. The harvested fluids can be titrated on mammalian cell monolayers, such as from tamarin monkeys, by RT-PCR-based assay or plaque assay. Growth comparison of inoculum versus recovered virus reveals whether the original phenotype of the chimeric virus is maintained in animals at early and late times in the course of infection. Wild-type viruses usually do not arise in the inoculated animals because a chimeric virus genome preferably contains a deletion of an entire GBV-B gene, which is replaced by and dependent upon an HCV gene. In any case, if an HCV gene-minus-virus results, it may be of significant interest for further study. The genetic changes underlying the restoration of the enhanced growth ability in the “revertant” virus can be determined by nucleotide sequence analysis, e.g., by RT-PCR amplification of the chimeric region. Although chimeric mutants are generally more stable than base substitutions, in some cases revertants or deletions arise at unexpectedly high frequency.

The secondary assays described above for in vitro cell culture assays can be adapted to evaluate activity of a test compound in vitro, using suitable animals (instead of host cells) that are susceptible to infection by a chimeric virus based on the tropism of GBV-B. Small new world monkeys, such as tamarins (Saguinus species), are suitable for this purpose. A compound that protects animals from infection by a chimeric virus, and particularly from pathogenicity of an infectious virus, would be an attractive candidate for developing an agent for treatment of HCV. Moreover, the animal models also provide a platform for pharmacokinetic and toxicology studies of a candidate compound.

Methods for testing drug efficacy are well known in the art, and established or new protocols can be used with a chimeric virus of the invention to determine efficacy of a candidate HCV inhibitor compound. The following methods for testing the efficacy of compounds as inhibitors of HCV in vivo are provided by way of example and are not intended to limit the invention.

For example, tamarins (Sagunus species) can be infected with a chimeric virus of the invention to the extent desired by the practitioner, e.g., readily detectable viremia, acute or chronic hepatitis conditions, etc. Animals infected with a chimeric virus of the invention can be divided into a test group (treated with a test compound using a desired dosage and according to standard protocols used in the art for this purpose) and a control group (not treated with test compound or treated with a placebo or sham compound). If desirable, a positive control may also be employed, such as administering a compound known to inhibit the HCV protein or chimeric HCV/GBV-B protein inserted into the chimeric virus. Blood samples may be withdrawn to determine the effect of the test compound on viral load. A reduction in viral load in previously infected animals treated with test compound, as compared with previously infected animals without treatment or treated with a placebo, would indicate that the compound inhibits HCV.

Tissue studies may also be performed using tissue samples, e.g., from liver, and assessing the pathogenesis or disease progression in the groups of infected animals by comparing the extent or stage of tissue damage. A slowed rate of disease progression or remission of disease in animals treated with the compound versus control animals would indicate that the compound inhibits HCV.

### EXAMPLES

The following Examples are provided for purpose of demonstrating certain aspects of the invention, and are not provided by way of limitation.

### Example 1

### Chimeric HCV/GBV-B NS 3 and NS4A Cofactor Requirements

The NS3 serine protease of GBV-B shares similar substrate specificity with HCV NS3. Since GBV-B is closely related to HCV and causes acute hepatitis in tamarins (Saguinus species), it was an attractive surrogate virus for in vivo testing of anti-HCV inhibitors in a small monkey model. In this Example, the protease activity of a full-length GBV-B NS3 protein and its cofactor requirement, is further characterized using in vitro translated GBV-B substrates.

Cleavages at the NS4A/4B and NS5A/5B junctions were readily detectable, but only in the presence of a minimum cofactor peptide derived from the central region of GBV-B NS4A (between amino acids Phe22 and Val36). Surprisingly, the GBV-B substrates were also cleaved by HCV NS3 protease in an HCV NS4A cofactor-dependent manner. These results confirmed that HCV and GBV-B share similar NS3 protease specificity while retaining a viral-specific cofactor requirement, and is consistent with a lack of sequence homology in the NS4A cofactor regions of HCV and GBV-B. A chimeric HCV/GBV-B bi-functional NS3, containing an N-terminal HCV protease domain and a C-terminal GBV-B RNA helicase domain, retained enzymatic activity in both domains, to permit the development of chimeric HCV/GBV-B that depends on HCV NS3 protease function.

### Materials and Methods

Cloning of expression plasmids. A full-length GBV-B NS3 cDNA was isolated directly from infected tamarin serum as described previously [Zhong et al, Virology 261: 216-226 (1999)].

Genes containing the GBV-B junction sites were isolated by PCR from cDNA clones of GBV-B (see FIG. 2). Any substrate for NS3 can be constructed as long as a junction site recognized by the NS3 protease is present. The primers used to amplify genes containing the NS4A/4B junction site were: 5′-ATATGGATCCGGTGCTACTGTCGCCCCAGTG-3′ (SEQ ID NO: 9) and 5′-ATATAAGCTTCACTTGGACGCAATTGCGCCTCC-3′ (SEQ ID NO: 10). The resulting PCR fragment was directly cloned into pET-28a (Novagen) between BamHI and HindIII. The primers used to amplify and clone the GBV-B NS4B/5A junction site were: 5′-AAATGGCTAGCGGTGAGTGGCCCACTATGGA-3′ (SEQ ID NO: 11) and 5′-ATATGGATCCCATGCGCACACCAGGTGTGTG-3′ (SEQ ID NO: 12). The PCR amplified fragment was cloned into pET/NS5BACT-His [Ferrari et al., J. Virol. 73: 1649-1654 (1999)] by replacing the NS5B coding region with an NS4B/5A substrate between NheI and BamHI sites.

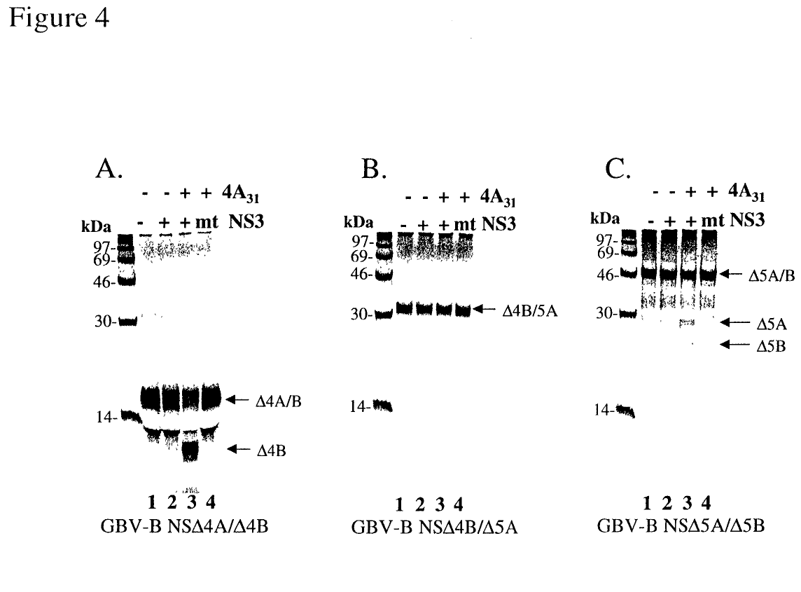

The GBV-B NS5A/5B junction site was cloned similarly to that of NS4B/5A using the primers: 5′-AAATGGCTAGCCAACTTAATTTGCGTGATGCAC-3′ (SEQ ID NO: 13) and 5′-ATATGGATCCCATCTTCTCAACACATCTCATTTC-3′ (SEQ ID NO: 14). The GBV-B coding regions shown in FIG. 4 were numbered according to the published amino acid positions [Simons et al., Proc. Natl. Acad. Sci. USA 92: 3401-3405 (1995); Muerhoff et al., J. Virol. 69: 5621-5630 (1995)]. Plasmids pNBNae, pJB1003, and pTS102 encoding the HCV NS3 junction sites NS4A/4B, NS4B/5A, and NS5A/5B, respectively, were cloned as described in Butkiewicz et al., supra.

A chimeric HCV/GBV-B NS3 was prepared by joining two PCR fragments: one containing an HCV-1a (H) NS3 protease coding region (aa 1-190), and the other containing a GBV-B RNA helicase coding region (aa 191-620). The resulting chimeric NS3 cDNA was cloned into pET/NS3-His (described in Zhong et al., supra) by excising the full-length GBV-B NS3 and inserting the chimeric cDNA between Nhe I and Bam HI sites.

A full-length HCV NS3 was isolated by PCR from pBRTM/HCV (1 to 3011), kindly provided by Dr. Charles M. Rice at Washington University, St. Louis, and cloned into the BamHI site in pQE40 (Qiagen,Valencia, Calif.). A His-tag and an enterokinase cleavage site (MRGSHHHHHHGSDYKDDDDKA) (SEQ ID NO: 15) were inserted at the amino terminus of the NS3 protein to facilitate its purification and the removal of the His-tag.

All plasmids were sequenced, and the coding regions (FIG. 4) were verified using an ABI Prism 377 DNA sequencer with XL upgraded gels (PE Applied Biosystems, Foster City, Calif.).

Expression andpurification. The full-length GBV-B NS3 and HCV/GBV-B chimeric NS3 were transformed into E. coli host strain JM109(DE3) (Promega). HCV NS3 was transformed into E. coli host strain M15(pREP4) (Qiagen). The transformed cells were grown in LB media to an O.D. value of 0.6 at 600 nM wavelength. Protein production was induced by 0.2 mM isopropylthio-β-D-galactoside (IPTG) at 24° C. for 4 hours. Each protein (GBV-B NS3-His, HCV/GBV-B NS3-His and HCV His-NS3) was purified similarly using a nickel chelated (Ni-NTA) affinity column, followed by passage through a Superdex 200 gel filtration column (Amershm Pharmacia Biotech, Piscataway, N.J.) according to Zhong et al., 1999, supra. The purity for each protein was greater than 95%. The final protein concentration was determined by a Bradford protein assay (Bio-Rad Laboratories, Hercules, Calif.) according to the manufacturer's instructions. Protein was stored in small aliquots at −70° C. in buffer containing 10% glycerol and 5 mM dithiothreitol (DTT).

In vitro translation of GBV-B and HCV substrates. Plasmids pNBNae, pJB1003, and pTS102 encoding the HCV NS3 junction sites NS4A/4B, NS4B/5A and NS5A/5B, respectively, were linearized as described by Butkiewicz et al., 1996, supra. Plasmids containing the junction sites GBV-B NS4A/4B, NS4B/5A, and NS5A/5B were linearized with Xho 1. All plasmids were in vitro transcribed with T7 RNA polymerase. In vitro-transcribed RNAs were translated in rabbit reticulocyte lysates (Promega, Madison, Wis.) in the presence of ³⁵S-methionine (Amersham Pharmacia Biotech) at 30° C. for 1 hr, according to the manufacturer's recommendation. All translation reactions were terminated by adding DNase-free RNase (Boehringer Mannheim, Indianapolis, Ind.) and cycloheximide for 15 min at 30° C.

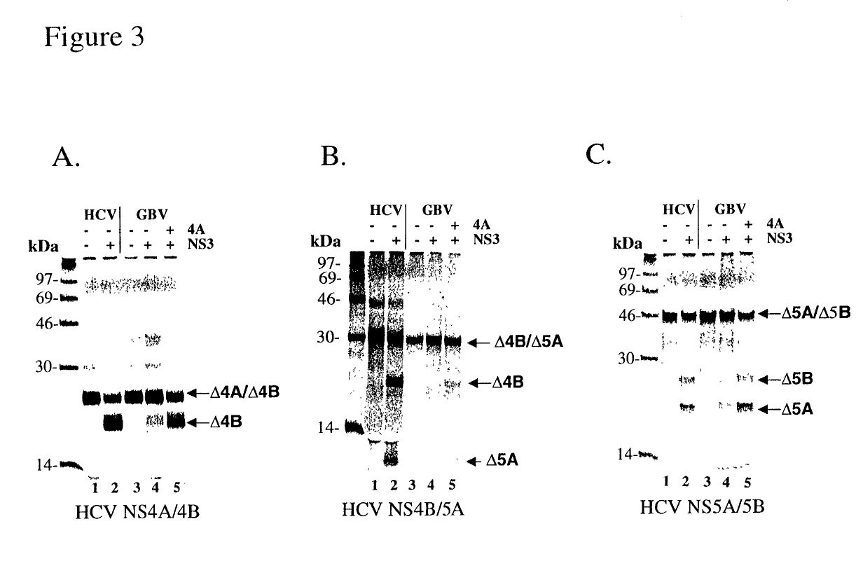

Trans-cleavage translation assays. To test GBV-B NS3/4A protease activity on HCV substrates, standard translation assays for NS3 protease were performed. Using a 20 μl reaction volume, purified GBV-B protease in a mixture containing 50 mM MOPS pH 7.5, 50 mM NaCl, 0.1% lauryl maltoside, 10% glycerol and 1 mM DTT was added to 2 μl of ³⁵S-labeled translated substrate from HCV (Δ4A/Δ4B, Δ4B/Δ5A, or Δ5A/Δ5B) to initiate the cleavage reaction. Labeled substrates and purified GBV-B protease were incubated in the presence or absence of 20 μM GBV-B NS4A peptide (aa 16-46, Δ4A₃₁). Final concentrations of GBV-B protease were 250 nM for NS4A/4B and 5 μM for NS4B/5A and NS5A/5B. HCV protease activity was assayed similarly in a mixture containing 50 mM MOPS pH 7.5, 300 mM NaCl, 0.1% NP40, 10% glycerol and 1 mM DTT. FIG. 3 shows the results of this experiment.

The in vitro translation experiment was repeated using ³⁵S-labeled substrates from GBV-B junction sites (Δ4A/Δ4B, Δ4B/Δ5A, and Δ5A/Δ5B) mixed with 2 μM of purified GBV-B protease in the presence or absence of 20 μM GBV-B NS4A peptide (Δ4A₃₁). A mutant (mt; NS3_{S137A}) GBV-B protease was also tested. The results are shown in FIG. 4.

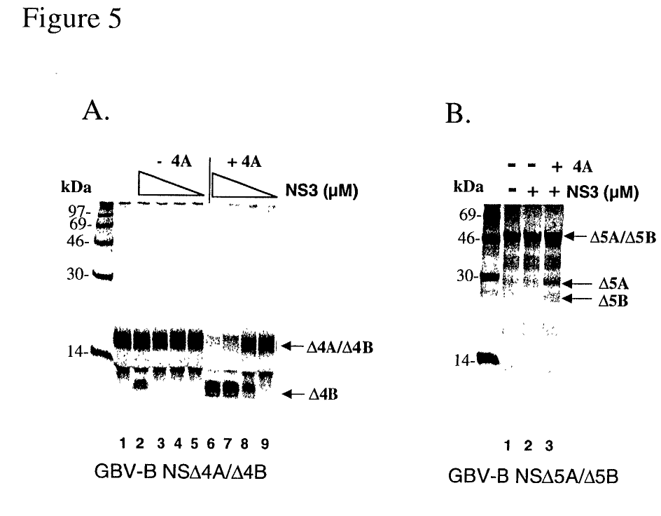

To test HCV NS3/4A protease activity on GBV-B substrates, purified full-length HCV NS3 protease domain (0 to 10 μM) was mixed with translated GBV-B substrates (Δ4A/Δ4B and Δ5A/Δ5B) in the presence or absence of 100 μM HCV NS4A cofactor peptide (Δ4A₁₃). The results are shown in FIG. 5.

To characterize GBV-B NS4A cofactor activity, ³⁵S-labeled GBV-B NS4A/4B substrate (Δ4A/Δ4B) was incubated with 1.5 μM GBV-B protease in the presence of various GBV-B NS4A peptides shown in FIG. 6. Cleavage products were then analyzed and detected by phosphorimaging and quantified by ImageQuant software (Molecular Dynamics, San Diego, Calif.). The cofactor activity was graded by comparing the enhancement of the protease activity to backgound activity (cleavage activity in the absence of NS4A cofactor): “+++”, 11-20 fold; “++”, 5-10 fold; “+”, 2.5-5 fold; “+”1.5-2 fold; and “−”, <1.5 fold of the background activity.

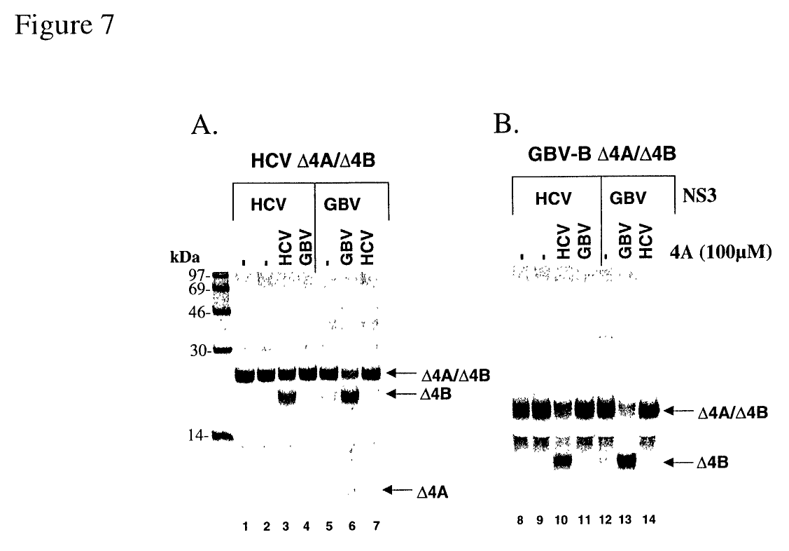

To determine the specificity of the NS4A cofactor for NS3, HCV or GBV-B NS3 protease was tested in the absence of NS4A peptide or in the presence of either HCV NS4A (Δ4A₁₃) or GBV-B NS4A (Δ4A₁₇) at 100 μM concentration. The assay was performed using the NS4A/4B substrates from either HCV (FIG. 7, panel A) or GBV-B (FIG. 7, panel B). HCV NS3 was tested at 50 nM for both substrates; GBV-B protease was tested at 250 nM for HCV substrate and at 2 μM for GBV-B substrate, respectively.

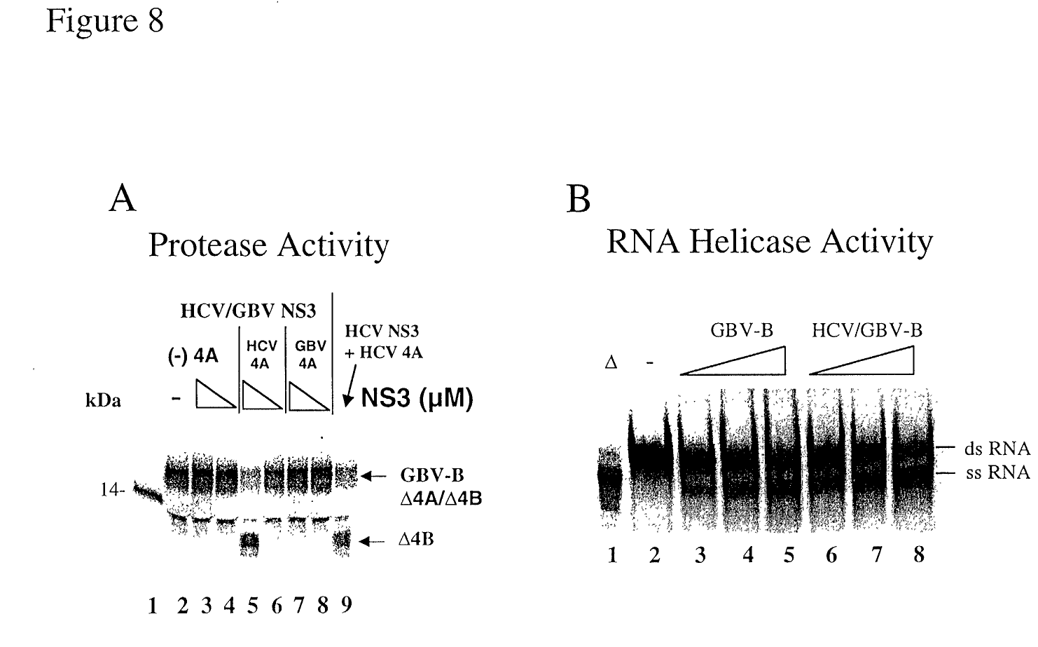

To further test the activity of a chimeric HCV/GBV NS3 (prepared as described above), 2 μM (FIG. 8, Panel A, lanes 3, 5, and 7) or 0.07 μM (FIG. 8, Panel A, lanes 4, 6, and 8) of the chimeric NS3 was mixed with a GBV-B NS4A/4B (Δ4A/Δ4B) substrate, in the absence or presence of 100 μM HCV NS4A cofactor (Δ4A₁₃) or GBV-B NS4A cofactor (Δ4A₁₇). Helicase activity was also tested as described below. The results are shown in FIG. 8.

All reactions were incubated for 1-2 hrs at 30° C., and terninated by adding an equal volume of 2× Laemmli sample buffer and boiling for 3 min. The cleavage products were separated by SDS-PAGE on a 15% gel, and detected and quantified by autoradiography.

Preparation ofsubstratesfor RNA helicase assay. RNA helicase substrates containing two complementary RNA strands were prepared as standard 5′/3′ substrates, annealed and gel purified (see, e.g., Kim et al., Biochem. Biophys. Res. Commun. 215: 160-166 (1995); Lee and Hurwitz, J. Biol. Chem. 267:4398-4407 (1992); Warrener and Collett, J. Virol. 69:1720-1726 (1995) for methods for preparing helicase substrate; note that any RNA sequences can be used provided a duplex can be formed). First, a pGEM-1 and pSP65 (both of Promega, Madison, Wis.) were each linearized using Pvu II and Xba I, respectively. The linearized plasmids were then transcribed using SP6 RNA polymerase to generate a 98-base RNA strand (upper strand) for pGEM-1 and a 38-base strand (lower strand) for pSP65. Both strands were separately transcribed in vitro using the bacteriophage SP6 or T7 RNA polymerase (Promega) in accordance with the manufacturer's instructions. The two RNA strands, which contain a 29-base complementary region, were annealed and gel purified. The annealed dsRNA substrates were end-labeled using γ-³³P-ATP and T4 polynucleotide kinase (Amersham Pharmacia Biotech). Alternatively, the in vitro transcribed RNAs have been labeled by incorporating α-³³P-CTP and gel purified.

RNA helicase assay. To compare the RNA helicase activity of a chimeric NS3 with native GBV-B NS3, a 20 μl reaction volume containing 2, 5 or 10 pmoles of a chimeric and wild type NS3 protein was tested for double-stranded RNA (dsRNA) unwinding activity in a standard RNA helicase assay. Fifty fmoles of labeled dsRNA substrates, prepared as described above, were added to the reaction buffer [50 mM Tris-HCl pH 7, 1 mM MgCl₂, 2 mM ATP, 2 mM DTT, 0.1 mg/ml of bovine serum albumin (BSA) and 4 units of RNAsin RNase inhibitor (Promega)]. The reaction mixture was incubated at 37° C. for 1 hour and then terminated by addition of 5 μl of stop buffer (100 mM Hepes pH 8, 20 mM EDTA, 0.1% NP-40, 30% glycerol and 0.3% bromophenol blue). The RNA products were electrophoresed on a polyacrylamide-TBE gel (Novex, San Diego, Calif.). The gel was dried and [³³P]-labeled release strands were detected by autoradiography and quantified by phosphorimaging.

Computer modeling of GBV-B NS3/4A interaction. To further understand how the GBV-B cofactor peptide interacts with the protease and enhances, the HCV NS3 protease structure was used as a template for modeling a GBV-B protease structure. The QUANTA program (Molecular Simulations Inc., San Diego, Calif.) was used for this purpose. The primary sequences of the NS3 protease domain between HCV and GBV-B share approximately 30% identity, whereas the full-length NS3 proteins share 40% identity. The secondary structure elements of the GBV-B protease model were transferred from existing HCV NS3 protease crystal structures [Kim et al., Cell 87:343-355 (1996); Yan et al., Protein Sci. 7: 837-847 (1998); Love et al., Clin. Diagn. Virol. 10:151-156 (1998)]. Side chain atom coordinates of HCV were adopted to model and position those in the corresponding GBV-B protease. The unresolved side chains were constructed using the CHARM program (Molecular Simulations Inc., San Diego, Calif.), and the newly modeled molecule relaxed to yield a smooth backbone with reasonable packing. The first 11 amino acids from the amino terminus were removed from the crystal structure to view the NS3/4A interaction.

### Results

A computer model shows that, compared with HCV, GBV-B has a similar substrate recognition site (SI pocket) next to the scissile bond for a small P1 residue (Cys). Furthermore, the amino acid that defines the S1 pocket specificity (Phe154) is conserved between HCV and GBV-B [Scarselli, J. Virol. 71: 4985-4989 (1997)]. It has been shown previously that the catalytic domain of a GBV-B NS3 protease is able to recognize and process the junction sites in an HCV polyprotein [Scarselli et al., supra]. Surprisingly, the GBV-B NS3 protease activity does not require a cofactor for activity, which is a hallmark of flavi-like viruses [Belyaev et al., J. Virol. 72: 868-872 (1998); Failla et al., J. Virol. 68: 3753-3760 (1994); Lin et al., supra; Tanji et al., J. Virol. 69: 1575-1581 (1995); Tomei et al., supra; Wiskerchen and Collett, Virol. 184: 341-350 (1991); Falgout et al., J. Virol. 65: 2467-2475 (1991)]. A lack of cofactor dependency may have been due to use of the catalytic domain to characterize the NS3 protease activity, which could have different activity than a full-length NS3, as was previously shown in HCV [Gallinari et al., J. Virol. 72: 6758-6769 (1998)]. Therefore, to prepare a chimeric virus, which has a GBV-B backbone and depends on an HCV protease, it was important to further characterize GBV-B protease activity using a full-length GBV-B NS3.

Cleavage of HCV polyprotein junction sites by GB V-B NS3 depends on a cofactor. The role of GBV-B NS4A in GBV-B-mediated trans-cleavages was previously studied using in vitro translated polyprotein substrates that contain the junction sites of HCV NS4A/4B, NS4B/5A, and NS5A/5B. A 31-amino acid peptide (aa 16-46, Δ4A₃₁) from the central region of GBV-B NS4A was arbitrarily chosen to test whether the GBV-B NS3 protease required a cofactor for full functional activity, such as the HCV NS4A cofactor required by the HCV protease. The in vitro translated substrates, as described previously by Butkiewicz et al., 1996, supra, were incubated with the full length GBV-B NS3 protease in the presence or absence of the NS4A₃₁ peptide. The results shown in FIG. 3 demonstrate that processing by the GBV-B protease at the NS4A/4B (FIG. 3, panel A), NS4B/5A (FIG. 3, panel B) and NS5A/5B (FIG. 3, panel C) junction sites yielded little, if any, of the expected products in the absence of the NS4A peptide. However, substrate processing was greatly improved by the NS4A peptide (compare FIG. 3, panels A, B and C, lanes 4 and 5 from each), which suggests that, like HCV, GBV-B also has a cofactor requirement that is localized to the central region of NS4A.

As a marker for appropriate polyprotein processing in this experiment, the full-length HCV NS3₆₃₁/4A [described by Sali et al., Biochem. 37: 3392-3401 (1998)] was used to generate similar cleavage products (FIG. 3, lane 2 of panels A, B and C).

GBV-B NS3 protease processes the GBV-B polyprotein at junction sites. Several plasmids encoding potential GBV-B substrate junction sites at NS4A/4B, NS4B/5A and NS5A/5B were constructed (FIG. 2) to evaluate homologous processing by GBV-B NS3. In vitro trans cleavage assays using these translated substrates were performed similarly to that described by Butkiewicz, 1996, supra. In these experiments a mutant GBV-B NS3 protease, in which the predicted active site serine 139 is replaced by an alanine (S137>A), served as a control for the protease activity. The results in FIG. 4, panel A, lane 3 show that cleavage at the NS4A/NS4B junction by wild type GBV-B NS3 occurs only in the presence of the GBV-B NS4A cofactor (by evidence of an NS4B product with a predicted molecular mass of 10 kDa). Similarly, processing at the NS5A/NS5B junction is also NS4A cofactor-dependent, producing 22 kDa NS5A and 20 kDa NS5B products (FIG. 4, panel C). However, no processing was observed for the NS3_{S137>A} mutant protease (lane 4 in FIG. 4, panels A, B and C). The wild type GBV-B NS3 did not cleave the GBV-B NS4B/5A substrate, either with or without the NS4A cofactor (FIG. 4, panel B, lanes 2 and 3). This is not surprising given the poor efficiency in cleaving this junction in HCV [Butkiewicz et al., supra].

HCV NS4A cofactor-dependent cleavage of GBV-B substrates by HCV NS3 protease. Having shown GBV-B NS4A cofactor-dependent GBV-B NS3 protease activity on HCV as well as GBV-B substrates, the HCV NS3 protease processing of heterologous GBV-B polyprotein junction sites was also tested. In these studies, a full-length HCV NS3₆₃, was expressed in E. coli and purified to greater than 95% homogeneity. Activity was tested for proteolytic processing in the presence and absence of a 13 amino acid cofactor peptide from HCV NS4A (amino acid 22 to 34), which is sufficient for cofactor activation of NS3 protease. The results in FIG. 5, panel A show dose-dependent and NS4A cofactor-enhanced cleavage activity at the NS4A/4B junction site (lanes 6 to 9). Interestingly, in the absence of NS4A cofactor, some processing occurred with a high concentration of HCV protease at 2.5 μM, which was the highest concentration tested (FIG. 5, panel A, lane 2). Cleavage of the GBV-B NS5A/5B site was inefficient and required 10 μM of HCV NS3 protease and was cofactor dependent (FIG. 5, panel B, lanes 2 and 3). No processing was observed for the predicted NS4B/5A site (data not shown). To map the exact junction site, several synthetic peptides containing the predicted cleavage sites were generated. HPLC and Mass Spectrum analyses of the cleavage products confirmed that Cys is the P1 residue at the NS4A/4B junction (data not shown) as described previously [Scarselli et al., supra].

NS4A cofactor peptide activity is virus-specific. The results described above demonstrate that a full-length GBV-B NS3 protease can trans-cleave GBV-B substrates and heterologous HCV substrates. Likewise, a full-length HCV NS3 protease can cleave heterologous GBV-B substrates. Since these cleavages are cofactor-dependent, it was of interest to determine whether the cofactor activity was virus-specific. For this purpose, the NS4A/4B substrates from both GBV-B and HCV were chosen to evaluate cofactor specificity. Each NS3 protease was tested using an HCV substrate or a heterologous substrate, in the presence of either the HCV NS4A cofactor peptide (4A₁₃; aa 22-34) or the GBV-B NS4A cofactor peptide (4A₁₇; amino acids 20-36). The results (shown in FIG. 7) demonstrate that there is a strict viral-specific cofactor requirement for each protease, so that protease activation is dependent upon an NS4A cofactor from the same virus (for HCV, compare lanes 3 and 10 with lanes 4 and 11; for GBV-B, compare lanes 6 and 13 with lanes 7 and 14). This finding is consistent with a lack of sequence homology between the GBV-B and HCV NS4A cofactor coding regions.

Mapping the minimal cofactor domain in GBV-B NS4A. The results above reveal that the GBV-B cofactor activity resides in the central region of NS4A from amino acids 16 to 46. To determine the minimum cofactor region required by GBV-B NS3 protease, a series of truncation peptides derived from the central region of GBV-B NS4A were synthesized. These synthetic peptides were tested for their ability to activate NS3 protease activity of GBV-B on the NS4A/4B junction site. As shown in FIG. 6, the C-terminal half (an 18-mer peptide corresponding to amino acids 29-46) of the central 31 amino acids did not possess the cofactor activity as it failed to enhance the protease activity. On the other hand, a 17-mer peptide derived from amino acid 20 to 36 of NS4A retained the cofactor activity. Further truncations from the N-terminus of this peptide produced a 15-mer peptide (amino acids 22-36), which is the minimum region that efficiently activates protease activity for trans-cleavage. Removal of additional residues from either terminus severely reduces cofactor activity. Interestingly, this minimum cofactor region in GBV-B NS4A (amino acids 22-36) overlaps with the central region of HCV NS4A (amino acids 22-33).

A chimeric NS3 retains both HCV protease and GB V-B RNA helicase functions. It has been shown through the inter-domain relationship between the protease and RNA helicase domains of NS3, by comparing the domain-derived activities with activity of a full-length NS3, that the enzymatic functions are relatively independent [Gallinari et al., supra; Gallinari et al., Biochem. 38: 5620-5632 (1999); Sali et al., supra; Howe et al., Protein Sci. 8: 1332-1341 (1999)]. This suggests that although the protease and RNA helicase domains are genetically linked, they may be functionally separated because a characterization of the full-length HCV NS3 shows two separated structural domains consisting of a protease and an RNA helicase linked together through a single stranded peptide. The full-length NS3/4A structure reveals similar characteristics observed in the domain structures [Yao et al., Structure 7: 1353-1363 (1999)].

A computer model for GBV-B NS3 was constructed, similarly as described, based on the full-length crystal structure of HCV NS3. This model reveals that the protease and helicase catalytic centers are segregated within the enzyme. The P-side of the substrate recognition site in the active site is occupied by the molecule's carboxy terminus. This provided a snapshot of the post cis-proteolytic processing at the NS3/4A junction. The most striking feature of the full-length GBV-B NS3 structure is that the protease and helicase domains are connected by a short solvent-exposed peptide. This flexible connecting strand allows both domains to adopt independent functional conformation optimal for each activity.

The unique inter-domain structural relationship in NS3 prompted a molecular “domain transplant”, in which the GBV-B NS3 protease domain was replaced by an analogous HCV NS3 protease domain. This chimeric NS3 contained an N-terminal HCV protease domain (amino acid 1-190) and a C-terminal GBV-B RNA helicase domain (amino acids 191-620). This NS3 was expressed in E. coli and purified. The solubility of the chimeric NS3 was comparable to that of native GBV-B NS3, indicating that the “xenografted” HCV protease domain did not affect proper folding.

Both the protease and the RNA helicase activities were characterized. The results in FIG. 8, panel A show that the chimeric NS3 retained protease activity by processing GBV-B substrate containing an NS4A/4B cleavage site. Protease activity required an HCV NS4A cofactor peptide (amino acids 22-34) (FIG. 8, panel A, lane 5), but was not functional using a GBV-B NS4A cofactor peptide (aa 20-36) (FIG. 8, panel A, lanes 7 and 8). A full-length HCV NS3 protease was used as a comparison, and also generated the cleavage products in an HCV NS4A cofactor-dependent manner (FIG. 8, panel A, lane 9). Furthermore, the chimeric NS3 had similar RNA helicase activity as native a GBV-B NS3 RNA helicase (FIG. 8, panel B). Thus, this chimeric NS3 is fully functional, having HCV protease activity and GBV-B RNA helicase activity.

### Discussion

This example shows that a GBV-B/tamarin model is biologically relevant to studying HCV. In addition, the characterization of GBV-B NS3 protease and helicase, and identification of a viral-specific NS4A cofactor requirement have allowed the development of a novel, in vivo chimeric HCV/GBV-B tamarin model. This improved model of the invention, contains a built-in HCV NS3 protease domain and NS4A cofactor region, or an HCV helicase domain, in a GBV-B genomic backbone, which replaces the homologous resident GBV-B proteins. The resulting chimeric virus is dependent upon HCV protease or helicase function.

Using a computer model of a full-length GBV-B NS3, precise protease domain swapping was achieved. The resulting chimeric HCV/GBV-B NS3 retained protease and RNA helicase activities comparable to the native GBV-B proteins. Mapping the GBV-B NS4A cofactor region provided the minimal peptide domain for insertion into a GBV-B genome to replace a GBV-B cofactor. Since HCV and GBV-B proteases share similar substrate specificity and process polyprotein from either virus, and have similar helicase activity, these findings allowed the development of HCV NS3 protease-dependent and HCV NS3 helicase-dependent GBV-B chimerics, which produce mature viral proteins that are competent of viral replication. Furthermore, the identification of a viral-specific cofactor requirement (FIG. 7) prompted a concomitant chimeric engineering in NS3 and NS4A to achieve efficient heterologous polyprotein processing by a chimeric virus. Such a chimeric virus is valuable for both in vitro and in vivo testing of anti-HCV inhibitors.

### Example 2

### Characterization of RNA-Dependent RNA Polymerase Activity Encoded by GBV-B NS5B

Replication of HCV, a positive-sense RNA virus, requires a viral-encoded RNA-dependent RNA polymerase (NS5B). Since viral RdRp is unique to virus-infected cells and essential to replication, it represents an attractive target for the design of antiviral agents to treat HCV-associated hepatitis. GBV-B NS5B contains sequence motifs characteristic of an RNA-dependent RNA polymerase. With its homology to HCV NS5B, GBV-B could serve as a surrogate assay system for studying HCV polymerase function, evaluating inhibitors against HCV RdRp, and developing anti-HCV drug therapies. An active GBV-B NS5B protein would help to further characterize polymerase activity of positive-sense RNA viruses, and enable the preparation of chimeric viruses based on a GBV-B genomic backbone, which are dependent on HCV RdRp activity. This Example characterizes GBV-B NS5B biochemically to demonstrate similarities between HCV and GBV-B RdRp, which enabled the development of the RdRp constructs and chimeric viruses of the present invention.

### Materials and Methods

Cells, plasmid. Bacterial strains employed in this Example include JM109 (DE3) (Promega), DH10B (GIBCO-BRL, Rockville, Md.), and XL-1 Blue (Stratagene, San Diego, Calif.). The bacterial expression vector was pET-28a (Novagen, Madison, Wis.) Cloning, expression andpurification of GBV-B NS5B protein. cDNA fragments containing the GBV-B NS5B region were isolated by reverse transcription coupled polymerase chain reaction (RT-PCR) from a pooled serum of GBV-B-infected tamarins (similarly as described in Example 1). The primers for RT-PCR were designed based on the published GBV-B sequence (GenBank Accession number: U22304) [Muerhoff et al., supra]. To facilitate cloning, and protein expression and purification, a methionine codon was introduced at the N-terminus for initiation of translation, and a six-histidine epitope tag (GSHHHHHH) was introduced at the C-terminus for affinity purification. Viral RNA was extracted from the serum using Trizol (GIBCO-BRL) according to the manufacturer's instructions. RT-PCR reactions were performed using the SuperScript one-step system (GIBCO-BRL). The amplified RT-PCR products were cloned into pET-28a (Novagen) between Nco I and Bam HI sites. A total of six independent clones were isolated and sequenced.

NS5B ACT19 was cloned into expression vector pET21 (Novagen) and protein expression was carried out in bacterial host JM109. Protein purification was performed under conditions similar to [Kim et al., supra]. Briefly, production of soluble GBV-B NS5B protein was induced in freshly transformed E.coli JM109 (DE3) cells, at O.D. value of 0.6, by isopropylthio-β-D-galactoside (IPTG) at a final concentration of 0.2 mM. After a 4-hour induction at 24° C., the cells were harvested and NS5B protein expression was confirmed by Western blot analysis. Soluble cell lysates were batch-adsorbed onto a nickel-chelated (Ni-NTA) column. After extensive washes (10 column volumes) under a high concentration of salt (1M NaCl), the protein was eluted off the column with a buffer containing 0.3 M imidazole. The protein was further purified using a Superdex-200 gel-filtration column (Amershm Pharmacia Biotech). Fractions from the gel-filtration column were separated using SDS-PAGE. The final protein concentration was determined by a Bradford protein assay (Bio-Rad Laboratories) according to the manufacturer's instructions. The protein was stored in small aliquots at −70° C. in the presence of 10% glycerol and 5 mM DTT.

Scintillation proximity assay (SPA). SPA was used to determine the standard activity of RdRp, and performed under standard conditions as described previously by Ferrari et al., supra. Briefly, reaction mixtures (50 μl in total volume) containing, in addition to other buffer components (20 mM HEPES, pH7.3, 7.5 mM DTT, 10 mM MnCl₂, 100 mM NaCl, 50 μg/ml BSA and 2 units of RNasin), 250 ng of poly(C), 25 ng of biotinylated oligo(G)₁₂, 5 μM GTP/ 0.1 μCi of ³H-GTP and 175 ng of GBV-B NS5B protein were incubated in a 96-well plate at room temperature for 3 hours. The reactions were terminated by addition of 50 μl of 100 mM EDTA in phosphate-buffered saline (PBS), the products were captured by SPA beads coated with streptavidin and ³H-GMP incorporation was determined with a scintillation counter.

Gel-based RdRp assay. Standard activity assays were performed in a reaction mixture (40 μl) containing 20 mM HEPES, pH 7.3, 15 mM MnCl₂, 100 μM of ATP, UTP, GTP and 10 μCi of α-³³P-CTP, 300 ng of purified protein and 0.2 μg of a 36-base synthetic RNA (5′-GGA₂₅UAUAUAUAU-3′). The sequence of the RNA was designed to form a stem-loop at the 3′-end to support the “copy-back” priming of RNA synthesis. The polymerization reaction was performed at 30° C. for 1 hr and terminated by phenol and chloroform extraction. RNA was ethanol precipitated and separated on a 15% TBE/6 M urea polyacrylamide gel (Novex). The gel was fixed, vacuum dried and subjected to autoradiography.

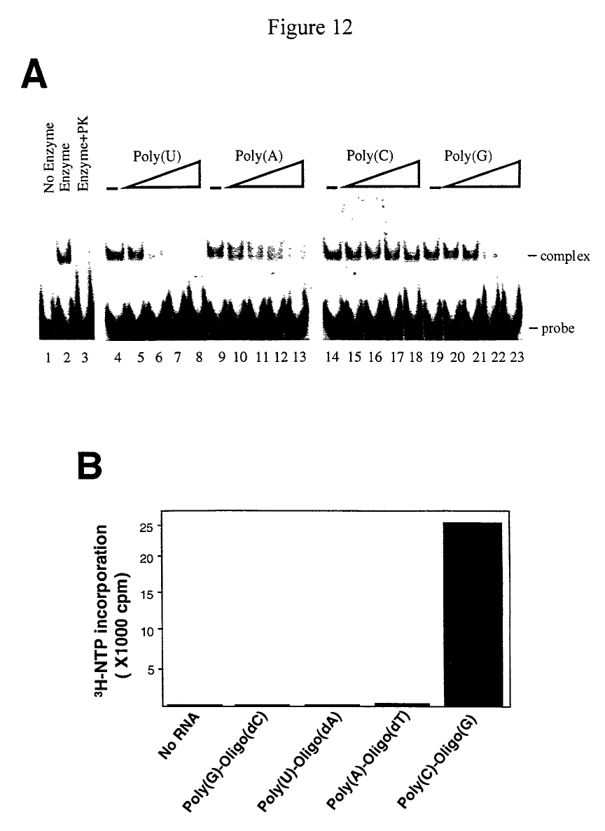

RNA-binding assay. The RNA binding assay was performed in a 10 μl reaction mixture containing 20 mM HEPES, pH 7.3, 7.5 mM MnCl₂, 7.5 mM DTT, 5% glycerol, 125 mM NaCl, 100 μg per ml of BSA, 1 unit of RNase inhibitor, 100 ng of GBV-B NS5B protein, 1 pmol of an end-labeled RNA probe with the sequence of 5′-U₂₈GGACUU CGGUCC-3′, and increasing amounts of homopolymeric RNA as depicted indicated in FIG. 12. The probe bound most tightly to the GBV-B NS5B among all the RNA probes of similar length tested (data not shown). Following incubation at room temperature for 30 min, the reaction mixture was separated on a nondenaturing 6% polyacrylamide gel at 180 volts for 1.25 hrs in 0.5×TBE buffer. The gel was dried and subjected to autoradiography.

### Results and Discussion

Expression and purification of GB V-B NS5B. Based on sequence alignment, a NS5B clone having a coding sequence identical to the published GBV-B NS5B sequence [Muerhoff et al., supra], representing the consensus sequence, was selected for subsequent protein expression and enzymatic characterization.

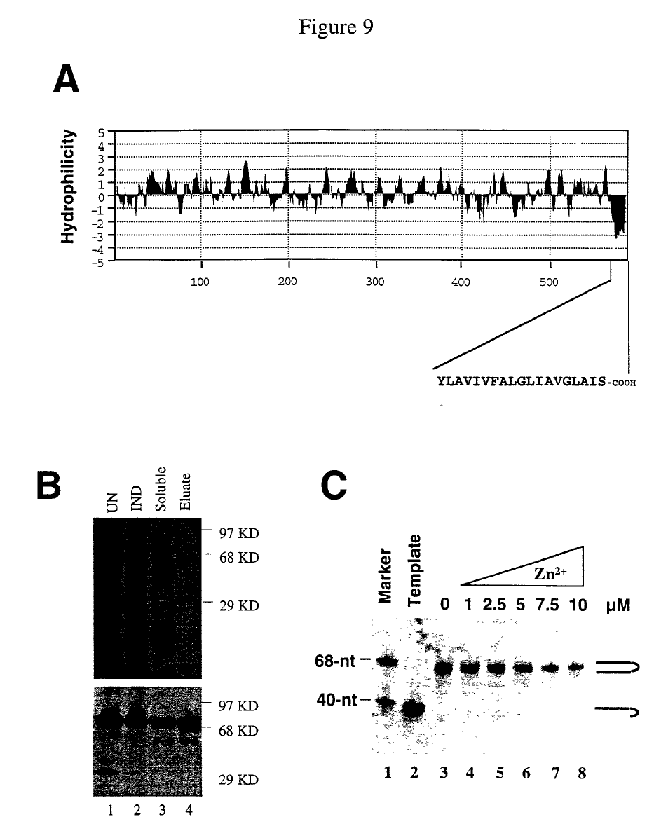

The initial attempt to express full-length GBV-B NS5B protein in E. coli proved rather problematic, mainly due to poor solubility of the protein in the host (data not shown). Prompted by our recent observation that removal of the C-terminal 21-amino acid hydrophobic domain of HCV NS5B significantly improved its solubility in E.coli [Ferrari et al., supra], a similar approach was adopted in this Example. As shown in FIG. 9, panel A, the hydropathy profile of GBV-B NS5B reveals the existence of a similar hydrophobic region (19 amino acids) at the C-terminus of the protein. Removal of the C-terminal 19 amino acids (NS5B ACT19) renders the protein soluble, which was purified using a nickel-chelated affinity (Ni-NTA) column (FIG. 9B, lanes 3 and 4). The final GBV-B NS5B ACT19 eluate was approximately 90% pure, and further used for enzymatic characterization.

GBV-B NS5B ΔCT19 possesses RdRp activity. To demonstrate that the purified NS5B ΔCT19 protein can direct RNA synthesis in vitro, a gel-based RdRp assay was established in which a 36-base synthetic RNA with a short stem-loop at the 3′-terminus was used as template. As shown in FIG. 9, panel C, the NS5B ΔCT19 extended the input RNA template and produced a near dimer-size product (lane 3). This result indicates that NS5B ΔCT19 is able to initiate RNA synthesis via a “copy-back” priming mechanism in which the 3′-terminal sequence of the RNA template folds back intramolecularly to form a stem-loop. This is similar to the in vitro “copy-back” activity (ability to catalyze nucleotidyl transfer by extending from the 3′ hydroxyl of a template, or from an RNA or DNA primer) observed for HCV [Behrens et al., supra; Lohmann et al., 1997, supra; Lohmann et al., Virol. 249: 108-118 (1998)]. FIG. 9, panel C also shows that Zn²⁺ metal ion inhibits GBV-B RdRp activity at low-micromolar (μM) concentrations (lanes 4 to 8). Further characterization of this inhibition is discussed the following section.

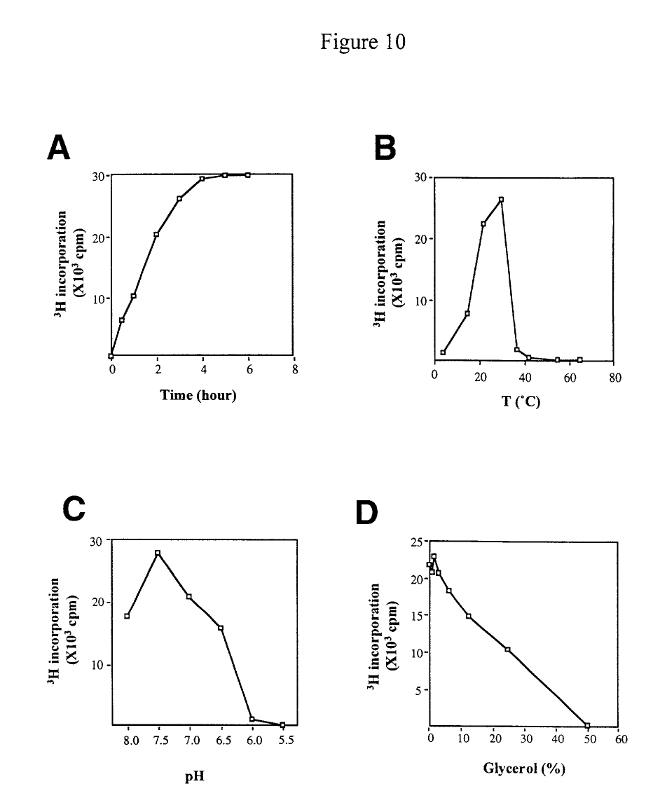

Reaction conditions for NS5B ΔCT19 RdRp activity. To further characterize the RdRp activity associated with the GBV-B NS5B protein, a scintillation proximity assay (SPA) was developed using polycytidylic acid (poly C) as the template and biotinylated oligoguanylic acid (oligoG₁₂) as the primer. Incorporation of ³H-GMP was measured after the polymerization products were captured onto the streptavidin-coated SPA beads [Ferrari, 1999, supra]. Optimization experiments showed that the optimal reaction time and temperature for the GBV-B RdRp is about 3 hours and 22-30° C., respectively (FIG. 10, panels A and B). The enzyme is active in a pH range of 6.5 to 8, with peak activity at about 7.3 (FIG. 10, panel C). Glycerol inhibits RdRp activity at concentrations higher than 3%. This finding is different than activity for a full-length HCV NS5B protein expressed in insect cells, which HCV activity required glycerol at higher concentrations (greater than 10%) for maximum activity [Lohmann, 1998, supra]. It is presumed that full-length recombinant HCV NS5B protein requires higher concentrations of glycerol to remain soluble due to a hydrophobic domain at the C-terminus.

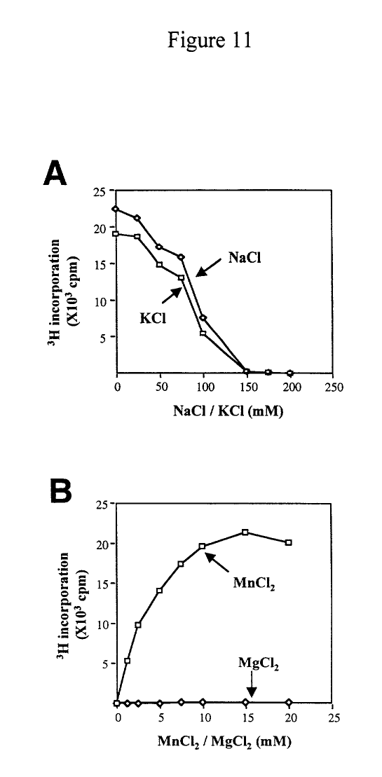

To determine the optimal concentrations of monovalent and divalent cations required for NS5B ΔCT19 enzymatic activity, an SPA-based RdRp assay was performed under conditions and titration of KCl, NaCl, MgCl₂, MnCl₂ or ZnCl₂. The RdRp activity was highest at low concentrations (0-25 mM) of monovalent salts (FIG. 11, panel A). The polymerase required divalent cation Mn²⁺ at an optimal concentration of 15 mM (FIG. 11, panel B). Surprisingly, Mg²⁺ could not replace Mn²⁺ to support the activity, even at concentrations as high as 20 mM (FIG. 11, panel B). This differs from HCV NS5B in that both Mn²⁻ and Mg²⁻ achieve comparable activity [Lohmann, 1998, supra].

Comparison of RNA-binding and RdRp activities. In addition to polymerase activity, viral RdRps must be able to bind RNA as a first step in the replication process. To demonstrate the RNA-binding activity associated with GBV-B NS5B protein, a gel-shift binding assay was performed using a radiolabeled 40-base synthetic RNA as probe (5′-U₂₈GGACUUCGGUCC-3′; SEQ ID NO: 16). This RNA probe was chosen for its high binding affinity to GBV-B NS5B (data not shown). FIG. 12, panel A shows that NS5B ΔCT19 binds to RNA, causing a bandshift of the probe (lane 2). An RNA-protein complex was not detected in lanes having no enzyme (lane 1) or after proteinase K treatment (lane 3).

To assess the binding affinity of NS5B ΔCT19 to various homopolymeric RNAs, competition experiments were performed with increasing concentrations of nonradiolabeled RNA homopolymers (0.1, 1, 10, 100×probe, respectively). FIG. 12, panel A, shows that poly (U) competes most efficiently for binding (lanes 5-8), followed by poly (G) (lanes 20-23), poly (A) (lanes 10-13), and poly (C) (lanes 15-18). These results demonstrate that NS5B ACT19 binds to RNA homopolymers with the affinity order of poly (U)>poly (G)>poly (A)>poly (C).

The ability of NS5B ΔCT19 to use RNA homopolymers as a template for RNA synthesis was also compared. FIG. 12, panel B shows that only poly(C)-oligo(G) supports polymerase activity, while poly(A)-oligo(dT), poly(U)-oligo(dA) and poly(G)-oligo(dC) are inactive. This result is similar to that observed for HCV RdRp [Lohmann, 1997, supra], suggesting an inverse correlation between RNA binding affinity and RdRp activity for both polymerases. It is consistent with the hypothesis that tight binding to an RNA template prevents the polymerase from translocating efficiently along the template and interferes with RNA elongation. As a weak binder, poly (A) is inactive for GBV-B NS5B (FIG. 12, panel B) but modestly active for HCV NS5B [Behrens, 1996, supra; Lohmann, 1997, sztpra]. This indicates that the GBV-B NS5B could bind to poly (A) more tightly than HCV NS5B.

Parallel enzymatic characterization revealed that GBV-B NS5B RdRp shared similar requirements with HCV NS5B for optimal activity. These similarities between RdRp activity of HCV and GBV-B NS5B are consistent with the phylogenetic proximity between HCV and GBV-B, thereby permitting the development of chimeric viruses having RdRp activity for use in a small monkey model.

The descriptions of the foregoing embodiments of the invention have been presented for purpose of illustration and description. They are not intended to be exhaustive or to limit the invention to the precise forms disclosed. Many modifications and variations can be made by those having skill in the art to suit the needs of the practioner without departing from the spirit of the invention. It is intended that the scope of the invention be defined by the claims appended hereto, and such modifications are intended to fall within the scope of the claims.

All polynucleotide lengths, peptide lengths, and molecular weight/mass values, are approximate, and are provided for description.

All patents, patent applications, publications, and other materials cited herein are hereby incorporated by reference in their entireties.

What is claimed is:

1. A nucleic acid encoding a GB virus-B (GBV-B) genome, wherein a nucleic acid sequence encoding a GBV-B NS3 protease is replaced with a nucleic acid sequence encoding a hepatitis C virus (HCV) NS3 protease, and a nucleic acid sequence encoding a GBV-B NS4A cofactor is replaced by a nucleic acid sequence encoding at least amino acids Cys22 to Gly34 of an HCV NS4A cofactor.

2. The nucleic acid of claim 1 which is a DNA.

3. The nucleic acid of claim 1 which is an infectious RNA.

4. The nucleic acid of claim 1, which encodes a polypeptide defined by SEQ ID NO: 5.

5. A cell transfected with the infectious RNA of claim 3.

6. A mammal infected with the infectious RNA of claim 3.

7. The mammal of claim 6 which is a new world monkey.

8. The mammal of claim 6 which is a tamarin (Saguinus species).

9. A chimeric HCV/GBV-B virus, comprising the nucleic acid of claim 1.

10. The chimeric HCV/GBV-B virus of claim 9, wherein the chimeric genome comprises a construct depicted in FIG. 1C.

11. The chimeric HCV/GBV-B of claim 9, which comprises a genome that encodes a polypeptide defined by SEQ ID NO: 5.

12. A cell infected with the chimeric HCV/GBV-B virus of claim 9.

13. A mammal infected with the chimeric HCV/GBV-B virus of claim 9.

14. The mammal of claim 13 which is a new world monkey.

15. The mammal of claim 13 which is a tamarin (Saguinus species).

16. A method for propagating an infectious, chimeric HCV/GBV-B virus, comprising culturing a cell of claim 5 under conditions that permit production of viable viruses in vitro.

17. The method of claim 16, wherein production of viable virus is detected by a viral quantification assay.

18. A method for propagating the chimeric HCV/GBV-B virus of claim 9, comprising infecting a mammal with the virus under conditions that permit production of viable viruses in vivo.

19. A method for screening for an inhibitor of an HCV NS3 protease and/or an HCV NS4A protease cofactor, comprising evaluating viral infection of the cells of claim 5 cultured under conditions that permit production of viable viruses, wherein a group of the cells is cultured in the presence of a candidate compound (test cells) and a different group of the cells is cultured in the absence of the candidate compound (control cells), and wherein a reduction in viral infection in test cells relative to control cells indicates that the compound inhibits the HCV NS3 protease and/or the HCV NS4A cofactor.

20. A method for evaluating infection and/or disease progression of a chimeric HCV/GBV-B virus in vivo, comprising monitoring viral load or disease progression, or both, in a mammal infected with the chimeric HCV/GBV-B virus of claim 9 under conditions that permit an infection by the chimeric HCV/GBV-B virus.

21. A method for screening for an inhibitor of HCV in vivo by inhibiting an HCV NS3 protease and/or an HCV NS4A protease cofactor, comprising monitoring viral load or disease progression, or both, in a mammal infected with a chimeric HCV/GBV-B virus of claim 9 under conditions that permit an infection by the chimeric HCV/GBV-B virus, wherein a group of the mammals is treated with a candidate compound (test group) and a different group of the mammals is treated with a placebo (control group), wherein a reduction in viral load or disease progression in the test group relative to the control group indicates that the compound inhibits HCV.

22. A nucleic acid encoding a GBV-B genome, wherein a nucleic acid sequence encoding a GBV-B NS5B RNA polymerase is replaced with a nucleic acid sequence encoding an HCV NS5B RNA dependent RNA polymerase.

23. The nucleic acid of claim 22 wherein the sequence encoding the HCV NS5B RNA dependent RNA polymerase is:
a palm domain of the HCV NS5B RNA polymerase;
a finger domain and a palm domain of the HCV NS5B RNA polymerase;
a palm domain and a thumb domain of the HCV NS5B RNA polymerase; or
a full length HCV NS5B RNA dependent RNA polymerase, wherein the finger domain is N-terminal and the thumb domain is C-terminal relative to the palm domain.

24. The nucleic acid of claim 22 which is a DNA.

25. The nucleic acid of claim 24 which is an infectious RNA.

26. The nucleic acid of claim 22, which encodes a polypeptide defined by SEQ ID NO: 6.

27. A cell transfected with the infectious RNA of claim 25.

28. A mammal infected with the infectious RNA of claim 25.

29. The mammal of claim 28 which is a new world monkey.

30. The mammal of claim 28 which is a tamarin (Saguinus species).

31. A chimeric HCV/GBV-B virus, comprising the nucleic acid of claim 22.

32. The chimeric HCV/GBV-B virus of claim 31, wherein the chimeric genome comprises a construct depicted in FIG. 1D.

33. The chimeric HCV/GBV-B of claim 31, which comprises a genome that encodes a polypeptide defined by SEQ ID NO: 6.

34. A cell infected with the chimeric HCV/GBV-B virus of claim 31.

35. A mammal infected with the chimeric HCV/GBV-B virus of claim 31.

36. The mammal of claim 35 which is a new world monkey.

37. The mammal of claim 35 which is a tamarin (Saguinus species).

38. A method for propagating an infectious, chimeric HCV/GBV-B virus, comprising culturing a cell of claim 27 under conditions that permit production of viable viruses in vivo.

39. The method of claim 38, wherein production of viable virus is detected by a viral quantification assay.

40. A method for propagating the chimeric HCV/GBV-B virus of claim 31, comprising infecting a mammal with the virus under conditions that permit production of viable viruses in vivo.

41. A method for screening for an inhibitor of an HCV NS5B RNA dependent RNA polymerase, comprising evaluating viral infection of the cells of claim 27 cultured under conditions that permit production of viable viruses, wherein a group of the cells is cultured in the presence of a candidate compound (test cells) and a different group of the cells is cultured in the absence of the candidate compound (control cells), and wherein a reduction in viral infection in test cells relative to control cells indicates that the compound inhibits the HCV NS5B RNA dependent RNA polymerase.

42. A method for evaluating infection and/or disease progression of a chimeric HCV/GBV-B virus in vivo, comprising monitoring viral load or disease progression, or both, in a mammal infected with a chimeric HCV/GBV-B virus of claim 31 under conditions that permit an infection by the chimeric HCV/GBV-B virus.

43. A method for screening for an inhibitor of HCV in vivo by inhibiting an HCV RNA dependent RNA polymerase, comprising monitoring viral load or disease progression, or both, in a mammal infected with a chimeric HCV/GBV-B virus of claim 31 under conditions that permit an infection by the chimeric HCV/GBV-B virus, wherein a group of the mammals is dosed with a candidate compound (test group) and a different group of the mammals is dosed with a placebo (control group), wherein a reduction in viral load or disease progression in the test group relative to the control group indicates that the compound inhibits HCV.

44. A nucleic acid encoding a GBV-B genome, wherein a nucleotide sequence encoding a GBV-B gene is replaced by a nucleotide sequence encoding a domain of an HCV gene which comprises functional activity, wherein the GBV-B gene and HCV gene are analogous.

45. A chimeric HCV/GBV-B virus, comprising the nucleic acid of claim 44.
